(19) United States
(12) Patent Application Publication
(10) Pub. No.: US 2010/0029551 A1
(43) Pub. Date: Feb. 4, 2010

(54) Treatment Of Inflammatory Lung Conditions With Aerosolized Macrolide Antibiotics

(76) Inventors: Howard V. Raff, Burlingame, CA (US); Ralph Niven, Burlingame, CA (US)

Correspondence Address: FENWICK & WEST LLP, SILICON VALLEY CENTER, 801 CALIFORNIA STREET, MOUNTAIN VIEW, CA, 94041, US

(21) Appl. No.: 12/533,779

(22) Filed: Jul. 31, 2009

Related U.S. Application Data
(60) Provisional application No. 61/085,363, filed on Jul. 31, 2008.

(51) Int. Cl. A61K 31/7048; A61P 29/00; A61K 38/13

(52) U.S. Cl. 514/11; 514/29

(57) ABSTRACT

Provided herein are pharmaceutical compositions comprising an anti-inflammatory macrolide compound suitable for aerosolized administration to the lungs by inhalation, and methods of treating inflammatory lung conditions, particularly those characterized by chronic inflammation, by administering an aerosolized anti-inflammatory macrolide compound to the lungs by inhalation. Also provided are unit dose formulations of an anti-inflammatory macrolide for aerosolized administration to the lungs by inhalation. Advantageously, the described methods and compositions allow for the targeted, localized delivery of anti-inflammatory macrolide compounds throughout the lungs without significant systemic absorption or deposition.

### CROSS REFERENCE TO RELATED APPLICATION

This application claims the benefit of U.S. provisional Application No. 61/085,363 filed Jul. 31, 2008, which is incorporated by reference in its entirety.

### FIELD

Provided herein are compositions and methods for the treatment of inflammatory lung conditions, including conditions characterized by chronic inflammation, by administering an aerosolized anti-inflammatory agent to the lungs by inhalation. The compositions and methods allow for more effective long-term treatment of chronic inflammation of the lungs, with minimal systemic side effects.

### BACKGROUND

The macrolide antibiotics are a family of broad-spectrum antibiotics characterized by a macrocyclic lactone ring structure having 14 to 16 atoms and typically at least one pendant sugar, amino sugar, or related moiety. Macrolide antibiotics are believed to inhibit bacterial protein synthesis by binding to the bacterial 50S ribosome and causing dissociation of peptidyl tRNA. Erythromycin, the first macrolide antibiotic, was discovered in 1952 and entered clinical use shortly thereafter. Erythromycin and its early derivatives typically have antibacterial activity against most gram positive bacteria, in particular streptococci, and are commonly used to treat a variety of respiratory infections.

Recent studies have revealed that many macrolide antibiotics, including most erythromycin A derivatives, have anti-inflammatory activity in addition to their antibacterial activity. The anti-inflammatory properties of macrolide antibiotics have been investigated in the treatment of several inflammatory lung conditions, such as cystic fibrosis (CF), chronic obstructive pulmonary disease (COPD), and chronic bronchitis. These studies typically involve oral administration of macrolide antibiotics at doses similar to those previously established as effective for anti-infective activity (e.g., doses associated with local concentrations >MIC₉₀ in targeted tissues).

While macrolide antibiotics are effective against a range of bacterial infections, they are also, like most broad spectrum antibiotics, associated with significant systemic side effects. For example, gastrointestinal (GI) disturbances, such as diarrhea, nausea, abdominal pain, and vomiting, are common at therapeutic oral doses of macrolide antibiotics (e.g., Brogden, Drugs, 48: 599-616 (1994); Langtry and Brogden, Drugs, 53: 973-1004 (1997)). In addition, oral macrolide antibiotics can alter the composition of flora in the bowel by selectively killing susceptible bacteria and allowing resistant species to flourish. In some instances, oral macrolides lead to development of Pseudomembranous colitis due to the macrolide-resistant bacterium Clostridium difficile. Such side effects are of even greater concern for treating chronic inflammatory conditions, which often require treatment for months to years.

U.S. Pat. Pub. No. 2004/0022740, to Baker et al., discloses aerosol compositions of macrolide antibiotics and methods of administering such compositions by inhalation to treat bacterial lung infections through administration of antibacterially effective amounts of a macrolide. Baker et al. does not discuss anti-inflammatory properties or uses of the macrolide antibiotics at doses lower than the antibacterially effective amounts, and further does not disclose administering the aerosolized formulations to treat inflammatory lung conditions.

Thus, there is a need in the art for compositions and methods that are safe and effective for use in treating inflammatory lung conditions, and in particular, for long-term treatment of chronic lung diseases, with minimal systemic side effects, low cost, and conduciveness to patient compliance.

### BRIEF SUMMARY

In one aspect, methods are provided for treating inflammation in the lungs of a subject by administering an effective amount of an aerosolized anti-inflammatory macrolide to the subject by inhalation, wherein the macrolide is administered with a dosing interval of at least 2 days, or preferably at least 3 days, or more preferably at least 5 days. In some aspects, the dosing interval is at least 7 days. In some aspects, the aerosolized anti-inflammatory macrolide is administered for at least 3 months.

In some preferred aspects, the effective amount is less than about 50 mg of the anti-inflammatory macrolide. In further aspects, the effective amount is less than about 45 mg, less than about 40 mg, less than about 35 mg, less than about 30 mg, less than about 25 mg, less than about 20 mg, less than about 15 mg, less than about 10 mg, or less than about 5 mg of the anti-inflammatory macrolide. In other aspects, the effective amount is greater than or equal to about 50 mg.

In some aspects, the effective amount treats the inflammation throughout the dosing interval without systemic side effects. In further aspects, the effective amount treats the inflammation throughout the dosing interval without nausea, vomiting, diarrhea, abdominal pain, or dyspepsia.

In certain aspects, the method further comprises administering a loading dose of an aerosolized anti-inflammatory macrolide to the subject by inhalation, the loading dose being administered with a dosing interval of less than 5 days. For example, in some aspects, the dosing interval between loading doses is between about 12 and 72 hours. In further aspects, the loading dose is greater than the effective dose.

In some aspects, the inflammation is induced by or associated with elevated levels of one or more cytokines in the lungs. For example, in some aspects, the inflammation is induced by or associated with elevated levels of a cytokine selected from the group consisting of: interleukin-1α (IL-1α), interleukin-1β (IL-1β), interleukin-8 (IL-8), interleukin-17 (IL-17), TNF-α, interferon-gamma (IFN-γ) and tissue growth factor β (TGF-β).

In further aspects, the inflammation is induced by or associated with increased levels of one or more inflammatory cells in the lungs. For example, in some aspects, the one or more inflammatory cells are selected from the group consisting of: eosinophils, lymphocytes, macrophages, neutrophils and monocytes.

In some aspects, the inflammation is associated with a condition selected from the group consisting of asthma, allergic asthma, emphysema, inflammatory lung injury, bronchiolitis obliterans (BO), pulmonary sarcoisosis, chronic obstructive pulmonary disease (COPD), interstitial lung disease, idiopathic pulmonary fibrosis, adult respiratory distress syndrome (ARDS), bronchiectasis, lung eosinophilia, interstitial fibrosis, and cystic fibrosis (CF). In further aspects, the inflammation is associated with transplantation of an organ, tissue and/or cells to the subject.

In yet further aspects, methods are provided for treating inflammation in the lungs of a subject, the methods comprising administering to the subject by inhalation a low dose of an aerosolized anti-inflammatory macrolide, wherein the low dose is less than 50 mg and effective to treat the inflammation without systemic side effects. In some aspects, the low dose is effective to treat the inflammation without nausea, vomiting, diarrhea, abdominal pain, vomiting, or dyspepsia.

In some aspects, the inflammation is induced by or associated with elevated levels of one or more cytokines in the lungs. In further aspects, the inflammation is induced by or associated with elevated levels of a cytokine selected from the group consisting of: interleukin-1α (IL-1α), interleukin-1β (IL-1β), interleukin-8 (IL-8), interleukin-17 (IL-17), TNF-α, interferon-gamma (IFN-γ) and tissue growth factor β (TGF-β).

In some aspects, the inflammation is induced by or associated with increased levels of one or more inflammatory cells in the lungs. For example, in some aspects, the one or more inflammatory cells are selected from the group consisting of: eosinophils, lymphocytes, macrophages, neutrophils and monocytes.

In various aspects, the inflammation is associated with a condition selected from the group consisting of asthma, allergic asthma, emphysema, inflammatory lung injury, bronchiolitis obliterans (BO), pulmonary sarcoisosis, chronic obstructive pulmonary disease (COPD), interstitial lung disease, idiopathic pulmonary fibrosis, adult respiratory distress syndrome (ARDS), bronchiectasis, lung eosinophilia, interstitial fibrosis, and cystic fibrosis (CF). In further aspects, the inflammation is associated with transplantation of an organ, tissue and/or cells to the subject.

In some aspects, the methods are effective in elevating levels of an anti-inflammatory cytokine in the lungs of the subject. For example, in some aspects, the methods are effective in elevating levels of an anti-inflammatory cytokine selected from interleukin-4 (IL-4) and interleukin-10. In further aspects, the methods are effective in reducing levels of a pro-inflammatory cytokine in the lungs of the subject. For example, in some aspects, the methods are effective in reducing the levels of a pro-inflammatory cytokine selected from the group consisting of: interleukin-1α (IL-1α), interleukin-1β (IL-1β), interleukin-2 (IL-2), interleukin-8 (IL-8), TNF-α, interferon-gamma (IFN-γ) and tissue growth factor β (TGF-β).

In various aspects, the anti-inflammatory macrolide is selected from the group consisting of: erythromycylamine, clarithromycin, azithromycin, dirithromycin, erythromycin A, and roxithromycin. In some preferred aspects, the anti-inflammatory macrolide is erythromycylamine. In other preferred aspects, the anti-inflammatory macrolide is azithromycin. In further preferred aspects, the anti-inflammatory macrolide is roxithromycin. In yet further preferred aspects, the anti-inflammatory macrolide is clarithromycin.

In further aspects, pharmaceutical compositions are provided comprising a combination of an anti-inflammatory macrolide and a calcineurin inhibitor, such as cyclosporine, in a pharmaceutically acceptable carrier suitable for aerosolized drug delivery, wherein the combination has a greater than additive effect on reducing or inhibiting inflammation in the lungs when administered by inhalation. In some aspects, the combination has a synergistic effect on reducing or inhibiting inflammation in the lungs when administered by inhalation.

In additional aspects, pharmaceutical compositions are provided for treating inflammation in the lungs of a subject, comprising one or more unit doses of an anti-inflammatory macrolide and a pharmaceutically acceptable carrier suitable for aerosolized administration of a unit dose of the anti-inflammatory macrolide to the lower respiratory tract of the subject using a nebulizer, a metered dose inhaler, or a dry powder inhaler. In some aspects, the unit dose is less than about 50 mg, less than about 45 mg, less than about 40 mg, less than about 35 mg, less than about 30 mg, less than about 25 mg, less than about 20 mg, less than about 15 mg, less than about 10 mg, or less than about 5 mg of the anti-inflammatory macrolide.

In yet further aspects, kits are provided comprising a container comprising one or more unit doses of an anti-inflammatory macrolide and a pharmaceutically acceptable carrier, wherein the container is fitted for use with a nebulizer, a metered dose inhaler, or a dry powder inhaler capable of aerosolizing and delivering a unit dose of the anti-inflammatory macrolide to the lower respiratory tract of the subject.

### BRIEF DESCRIPTION OF THE DRAWINGS

FIG. 1 shows plasma concentrations profile (N=3) of erythromycylamine in male Sprague Dawley rats following endotracheal administration. Dose concentration is 5 mg/kg free base.

FIG. 2 shows the concentration profile of erythromycylamine in whole lung (N=3) in male Sprague Dawley rats following endotracheal administration. Dose concentration is 5 mg/kg free base.

FIG. 3 shows the dose exposure and peak concentration-time profiles of erythromycylamine in male Sprague Dawley rats following endotracheal administration of erythromycylamine at 0, 48 and 96 h. Dose concentration is 5 mg/kg.

FIG. 4 shows the plasma concentrations (N=5) of erythromycylamine in male Sprague Dawley rats following endotracheal administration. Dose concentration is 5 mg/kg free base.

FIG. 5 shows the concentration of erythromycylamine in whole lung (N=5) in male Sprague Dawley rats following endotracheal administration. Dose concentration is 5 mg/kg free base.

FIG. 6 shows the concentration of erythromycylamine (N=5) in left lung lobes of male Sprague Dawley rats following endotracheal administration. Dose concentration is 5 mg/kg free base.

FIG. 7 shows the concentration of erythromycylamine (N=5) in accessory lung lobes of male Sprague Dawley rats following endotracheal administration. Dose concentration is 5 mg/kg free base.

FIG. 8 shows the concentration of erythromycylamine (N=5) in right cranial lobes of male Sprague Dawley rats following endotracheal administration. Dose concentration is 5 mg/kg free base.

FIG. 9 shows the concentration of erythromycylamine (N=5) in right middle lobes of male Sprague Dawley rats following endotracheal administration. Dose concentration is 5 mg/kg free base.

FIG. 10 shows the concentration of erythromycylamine (N=5) in right caudal lobes of male Sprague Dawley rats following endotracheal administration. Dose concentration is 5 mg/kg free base.

FIG. 11 shows a schematic of nose-only exposure system for rodents.

FIG. 12 shows total cell counts and differentials from LPS exposed F344 rats.

FIG. 13 shows erythromycylamine sulfate concentrations in lung tissues.

FIG. 14 shows particle distribution for erythromycylamine sulfate exposure atmosphere.

FIG. 15 shows cytokine response 4 hours post LPS exposure

FIG. 16 shows cytokine response 24 hours post LPS exposure

### DETAILED DESCRIPTION

The descriptions of various aspects of the invention are presented for purposes of illustration, and are not intended to be exhaustive or to limit the invention to the forms disclosed. Persons skilled in the relevant art can appreciate that many modifications and variations are possible in light of the aspect teachings.

It should be noted that the language used herein has been principally selected for readability and instructional purposes, and it may not have been selected to delineate or circumscribe the inventive subject matter. Accordingly, the disclosure is intended to be illustrative, but not limiting, of the scope of invention.

It must be noted that, as used in the specification, the singular forms “a,” “an” and “the” include plural referents unless the context clearly dictates otherwise.

It will be appreciated that the same thing may be said in more than one way. Consequently, alternative language and synonyms may be used for any one or more of the terms discussed herein. No significance is to be placed upon whether or not a term is elaborated or discussed herein. Some synonyms or substitutable methods, materials and the like are provided. Recital of one or a few synonyms or equivalents does not exclude use of other synonyms or equivalents, unless it is explicitly stated. Use of examples, including examples of terms, is for illustrative purposes only and does not limit the scope and meaning of the aspects of the invention herein.

Provided herein are methods of treating an inflammatory lung condition by administering an aerosolized anti-inflammatory macrolide to the lungs by inhalation. The “inflammatory lung condition” can be any disease, disorder, or condition characterized by an inflammatory response in the lungs and/or pulmonary inflammation.

In various aspects, administering an anti-inflammatory macrolide according to a method provided herein alleviates one or more symptoms of pulmonary inflammation and/or an inflammatory lung condition targeted for treatment. Examples of conditions that can be treated with methods and compositions provided herein include, but are not limited to, asthma, allergic asthma, emphysema, bronchitis, chronic bronchitis, pneumonia, inflammatory lung injury, bronchiolitis obliterans (BO), pulmonary sarcoisosis, chronic obstructive pulmonary disease (COPD), interstitial lung disease, idiopathic pulmonary fibrosis, adult respiratory distress syndrome (ARDS), bronchiectasis, lung eosinophilia, interstitial fibrosis, and cystic fibrosis (CF). However, methods and compositions provided herein are not limited to specific and/or recognized diseases or conditions, but rather can be used to treat any subject suffering from pulmonary inflammation or its effects. Characteristic symptoms of inflammatory lung conditions, including symptoms associated specifically with inflammation and methods for diagnosing and measuring such symptoms are known in the art.

In some aspects, methods are provided herein for treating a non-infectious inflammatory lung condition. For example, in some aspects, the non-infectious inflammatory lung condition is asthma, allergic asthma, emphysema, inflammatory lung injury, bronchiolitis obliterans (BO), pulmonary sarcoisosis, chronic obstructive pulmonary disease (COPD), interstitial lung disease, idiopathic pulmonary fibrosis, adult respiratory distress syndrome (ARDS), bronchiectasis, lung eosinophilia, interstitial fibrosis, and cystic fibrosis (CF).

As used herein, “treating” can include prevention, amelioration, alleviation, and/or elimination of an inflammatory lung condition and/or one or more symptoms of such condition, as well as improvement in the overall well being of a patient, as measured by objective and/or subjective criteria.

The term “subject” is understood to include all animals, including, but not limited to, humans, or veterinary subjects, such as other primates, dogs, cats, horses, cows, and the like. In some aspects, the subject does not suffer from a pulmonary infection. In some preferred aspects, the subject does not suffer from a pulmonary bacterial infection. For example, in certain aspects, the subject does not suffer from a pulmonary infection with Streptococcus pneumoniae, Haemophilus influenzae, Staphylococcus aureus, Moraxella catarrhalis, Legionella pneumonia, Chlamydia pneumoniae, and/or Mycoplasma pneumoniae.

In some aspects, the subject suffers from pulmonary inflammation associated with a causative agent or event other than infection, such as tissue or organ transplantation, physical trauma or exposure to irritants or pollutants. For example, in some aspects, the subject suffers from pulmonary inflammation associated with exposure to cigarette smoke, chemical fumes, fossil fuel exhaust, asbestos, silica dust, metal dust, bacterial dust, animal dusts, allergens (e.g., pollen, dust, cotton, cat dander) and/or other airborne particles. In some aspects, the subject suffers from a condition such as pigeon fancier's disease, farmer's lung, grain handler's lung, mushroom worker's lung, bagassosis, detergent worker's lung, maple bark stripper's lung, malt worker's lung, paprika splitter's lung, or bird breeder's lung.

In some aspects, the subject suffers or has suffered from an infection that produces lung scarring, such as tuberculosis, connective or collagen tissue diseases such as rheumatoid arthritis, systemic sclerosis and systemic lupus erythematosis, idiopathic pulmonary fibrosis, or pulmonary fibrosis of genetic/familial origin.

In some aspects, the subject does not currently suffer from pulmonary inflammation, but has suffered from pulmonary inflammation and/or is at risk of developing pulmonary inflammation and is administered an anti-inflammatory macrolide as a prophylactic measure or as part of a maintenance regimen. For example, in some aspects, the subject has a disease or condition, such as cystic fibrosis (CF), which predisposes the subject to pulmonary inflammation.

In other aspects, the subject suffers from pulmonary inflammation associated with a pulmonary bacterial infection but elimination of the infection (e.g., with an antibiotic) is insufficient to ameliorate the inflammation. Thus, in some aspects, a subject suffering from inflammation associated with a pulmonary bacterial infection is administered an anti-inflammatory macrolide until the bacterial infection is substantially eradicated and administration of the anti-inflammatory macrolide is continued to treat chronic or persistent inflammation that is unaccompanied by a bacterial infection.

In some aspects, the subject suffers from pulmonary inflammation associated with a viral infection, such as an infection by human rhinovirus, enterovirus, herpesvirus, coronavirus, influenza virus, parainfluenza virus, respiratory syncytial virus (RSV), cytomegalovirus (CMV), or adenovirus. For example, in some aspects, the subject suffers from ventilator associated pneumonia (VAP), hospital-acquired pneumonia (HAP), community acquired pneumonia (CAP), and/or another form of pneumonia.

In some aspects, the subject suffers from pulmonary inflammation associated with a fungal infection or a non-invasive fungus-induced mucositis caused by a fungus such as, but not limited to, Absidia, Aspergillus flavus, Aspergillus fumigatus, Aspergillus glaucus, Aspergillus nidulans, Aspergillus versicolor, Alternaria, Basidiobolus, Bipolaris, Candida albicans, Candida lypolytica, Candida parapsilosis, Cladosporium, Conidiobolus, Cunninahamella, Curvularia, Dreschlera, Exserohilum, Fusarium, Malbranchia, Paecilomvces, Penicillium, Pseudallescheria, Rhizopus, Schizophylum, and Sporothrix.

In yet further aspects, the subject suffers from pulmonary inflammation associated with interstitial lung disease, such as but not limited to, interstitial pneumonia (UIP), non-specific interstitial pneumonia (NSIP), diffuse alveolar damage (DAD), organizing pneumonia (OP), desquamative interstitial pneumonia (DIP), respiratory bronchiolitis (RB), or lymphocytic interstitial pneumonia (LIP).

In some aspects, the subject suffers from an underlying condition associated with pulmonary inflammation which can be exacerbated under one or more conditions, such as but not limited to, bacterial infection, viral infection, fungal infection or non-invasive fungus-induced mucositis, interstitial lung disease, allergies, tissue or organ transplantation, physical trauma, exposure to irritants, pollutants, or other airborne particles or fumes. In some preferred aspects, the underlying condition is associated with chronic pulmonary inflammation. In some aspects, the underlying condition is asthma, which may be exacerbated by allergens, exercise, pollution, cold, stress, viral infection, bacterial infection, fungal infection and/or other conditions.

An “anti-inflammatory macrolide” is a macrolide antibiotic capable of having an anti-inflammatory effect in the lungs. Compounds suitable for use as anti-inflammatory macrolides include azalides, such as erythromycin, EM703, dirithromycin, azithromycin, 9-dihydro-9-deoxo-9a-aza-9a-ho-moerythromycin, HMR 3004, HMR 3647, HMR 3787, josamycin, spiramycin, midecamycin, erythromycylamine, ABT 773, flurithromycin, clarithromycin, tylosin, tilmicosin, troleandomycin, oleandomycin, desmycosin, CP-163505, roxithromycin, miocamycin and rokitamycin, and pharmaceutically acceptable salts thereof. In some aspects, the anti-inflammatory macrolide is an analogue, derivative, metabolite, or prodrug of a macrolide described herein, such as a ketolide (e.g., a 3-ketone), a lactam (e.g., an 8a- or 9a-lactam) or a derivative lacking one or more of the sugar moieties.

Compounds described herein include all possible stereoisomers, and include racemic mixtures and individual enantiomers/diastereomers, substantially free of other enantiomers/diastereomers. Methods for synthesizing and isolating stereoisomers are known in the art. For example, methods for preparing 9(S)-erythromycylamine and 9(R)-erythromycylamine are described by Massey et al., Tetrahedron Letters, 157 (1970); Wildsmith, Tetrahedron Letters, 29 (1972); and Massey et al., J. Med. Chem., 17: 105-107 (1974).

In some preferred aspects, the anti-inflammatory macrolide antibiotic has a 14- or 15-member macrocyclic lactone ring, wherein “member” refers to the carbon atoms or heteroatoms in the ring. For example, in some aspects, the anti-inflammatory macrolide is a 14-member macrolide derivative of erythromycin A, such as clarithromycin, roxithromycin, erythromycylamine, or dirithromycin. Dirithromycin is a lipid soluble prodrug of erythromycylamine that is rapidly hydrolyzed in plasma (half-life of about 30 minutes) to form erythromycylamine.

In some preferred aspects, the anti-inflammatory macrolide is erythromycylamine. Erythromycylamine is highly unstable at low pH and is rapidly degraded to non-active metabolites in the gastrointestinal tract. The erythromycylamine prodrug dirithromycin is therefore preferred for oral administration. Advantageously, aerosolized administration according to methods provided herein substantially avoids acid-catalyzed degradation associated with the oral administration of erythromycylamine.

In further aspects, the anti-inflammatory macrolide is a derivative or analogue of erythromycylamine, such as but not limited to, a sulfonamide derivative of erythromycylamine (e.g., as described in U.S. Pat. No. 3,983,103); an N-substituted derivative of erythromycylamine (e.g., as described in Ryden et al., J. Med. Chem., 16: 1059 (1973) or U.S. Pat. No. 4,016,263); an erythromycylamine 11,12-carbonate compound (e.g., as described in U.S. Pat. No. 4,283,527); a 9-N-ethenyl derivative (e.g., as described in U.S. Pat. No. 5,854,219) or an aldehyde-erythromycylamine condensation product (e.g., as described in U.S. Pat. Nos. 3,681,322 and 4,048,306).

In some aspects, the anti-inflammatory macrolide is clarithromycin. Without being limited by a particular theory, the administration of aerosolized clarithromycin by inhalation is believed to be particularly advantageous relative to oral dosing, since the first-pass metabolism of clarithromycin produces a metabolite, 14-hydroxy clarithromycin, which is approximately twice as active as clarithromycin and has a significantly longer plasma half-life than clarithromycin. Advantageously, local administration of clarithromycin to the lungs by aerosolized inhalation largely avoids first pass metabolism and thereby minimizes systemic activity and associated systemic side effects.

In other preferred aspects, the anti-inflammatory macrolide is a 15-member macrolide, such as azithromycin or a derivative thereof. Derivatives of azithromycin having anti-inflammatory properties are described, e.g., in EP Pat. No. 0283055, which is herein incorporated by reference.

In some aspects, the anti-inflammatory macrolide is capable of forming drug micelles. Without being limited by any particular theory, it is believed that macrolide drug micelles can interact with phospholipids in the plasma and/or organellar membranes of cells of inflamed tissues and/or cells involved in mediating an inflammatory response.

In some aspects, the effect of an anti-inflammatory macrolide is detected as a reduction in the degree and/or incidence of inflammation in the lungs. Pulmonary inflammation can be observed directly, for example using chest x-rays, positron emission tomography (PET), HRCT, or other visual techniques, as erythema (redness), edema (swelling), denudation, bronchoconstriction, alveolitis, vasculitis, airway edema, mucous plug formation, airway remodeling, fibrosis and/or other physiological changes of lung parenchyma, mucosa, pleura, and/or other tissues.

Pulmonary inflammation can also be diagnosed by characteristic symptoms, including but not limited to, chest pain, shortness of breath (exertional dyspnea), airway obstruction, bronchospasm, bronchial hyper-reactivity, coughing, expectoration, wheezing, fever, and/or impairment of lung function. Thus, in some aspects, administering an anti-inflammatory macrolide according to methods provided herein reduces swelling in the lungs and/or alleviates one or more symptoms of pulmonary inflammation.

In some aspects, administering an anti-inflammatory macrolide according to a method provided herein reduces the incidence and/or severity of an inflammatory response (IR) in the lungs, as evidenced by one or more IR indicators. IR indicators include, but are not limited to, oxygen radicals (hydrogen peroxide), nitric oxide (NO), histamines, leukotrienes, prostaglandins, inflammatory cytokines, interleukins and chemokines. For example, in some preferred aspects, administering an anti-inflammatory macrolide reduces levels of one or more inflammatory cytokines in the lungs relative to levels associated with the condition targeted for treatment or an IR. Methods for sampling levels of cytokines and other factors in the lungs are well known in the art, and include, e.g., bronchoscopy with bronchoalveolar lavage (BAL), lung biopsy, sputum induction, and the like. For example, methods for testing sputum samples for inflammatory markers are described in Kim et al., Chest, 129: 1148-1154 (2006) and Sagel et al., Proc. Am. Thorac. Soc., 4: 406-417 (2007).

An “inflammatory cytokine” is a cytokine that promotes or modulates an IR, for example by exerting a pyrogenic effect, modulating the expression, synthesis, and/or secretion of an inflammatory cytokine or other mediator of inflammation, and/or modulating the proliferation, migration, adhesion, and/or infiltration of inflammatory cells. Inflammatory cytokines can exert modulatory effects against the cell from which they were secreted (autocrine effect) and/or surrounding cells (paracrine effect), including, for example, bronchiolar and alveolar endo/epithelium cells and inflammatory cells, such as alveolar macrophages (AM), polymorphonuclear cells (PMNs), and the like. Non-limiting examples of inflammatory cytokines include interleukin-1α (IL-1α), interleukin-1β (IL-1β), interleukin-2 (IL-2), interleukin-4 (IL-4), interleukin-6 (IL-6), interleukin-8 (IL-8), interleukin-10 (IL-10), interleukin-11 (IL-11), interleukin-12 (IL-12), interleukin-17 (IL-17), interleukin-18 (IL-18), regulated on activation, normal T expressed and secreted (RANTES), growth-regulated oncogene (GRO-KC), tissue necrosis factor-alpha (TNF-α), interferon-alpha (IFN-α), interferon-gamma (IFN-γ), leukocyte inhibitory factor (LIF), and tissue growth factor β (TGF-β).

Thus, in some aspects, administering an anti-inflammatory macrolide according to methods provided herein leads to reduced levels of one or more pro-inflammatory cytokines, such as interleukin-1α (IL-1α), interleukin-1β (IL-1β), interleukin-6 (IL-6), interleukin-8 (IL-8), interleukin-17 (IL-17), TNF-α, interferon-gamma (IFN-γ) and tissue growth factor β (TGF-β). In further aspects administering an anti-inflammatory macrolide according to methods provided herein leads to increased levels of one or more anti-inflammatory cytokines, such as interleukin-4 (IL-4), interleukin-10 (IL-10), interleukin-13 (IL-13), interferon-alpha (IFN-α), and tissue growth factor β (TGF-β).

In further aspects, administering an anti-inflammatory macrolide according to methods provided herein leads to an improvement in lung function, for example as evidenced by a pulmonary function test (PFT), such as expiratory flow rate, FEF 25-75, FEV1, or FVC. The “expiratory flow rate” is measured by inspiring maximally to total lung capacity and then exhaling as rapidly and as completely as possible into a spirometer, which measures the rate at which air is expelled from the lungs. “Forced expiratory flow 25-75” (FEF 25-75) is the expiratory flow rate over the midportion of a forced exhalation, and “forced expiratory volume in one second” (FEV1) is the maximum expiratory volume during a one-second interval of a forced expiratory flow test. The “forced vital capacity” (FVC) is the total volume of air resulting from a forced expiratory flow test. Combinations of the above measures can also be used as indicators of lung function. For example, the FEV1/FVC ratio is sometimes used as a measure of bronchoconstriction and airway obstruction.

In further aspects, administering an anti-inflammatory macrolide according to a method provided herein modulates expression of one or more diagnostic markers of inflammation or an IR, which may precede the manifestation of clinical symptoms. For example, in some aspects, an anti-inflammatory macrolide inhibits, inactivates, or otherwise modulates the transcription factor nuclear factor (NF)-κB, which modulates the expression of numerous IR indicators, including inducible nitric oxide synthase (iNOS), intercellular adhesion molecule-1 (ICAM-1), chemokines (e.g., IL-8), and inflammatory cytokines.

In some aspects, administering an anti-inflammatory macrolide decreases levels of one or more inflammatory cells in the lungs. “Inflammatory cells” are white blood cells (WBCs) that act as cellular mediators of IR/inflammation in the lungs, and include but are not limited to eosinophils, lymphocytes, macrophages, neutrophils and monocytes. The recruitment, proliferation, migration, adhesion, and/or infiltration of inflammatory cells, such as alveolar macrophages (AMs) or polymorphonuclear cells (PMNs), in the lungs and/or the state of such cells (e.g., whether activated) can be used as indicators of an IR and inflammation in the lungs. The activation of inflammatory cells can be measured, for example, by measuring the expression of one or more genes and/or the secretion of one or more soluble proteins, such as inflammatory mediators, that are indicative of activation.

Certain methods provided herein are based on the discovery that macrolides exert anti-inflammatory activity in the lungs at concentrations that are significantly lower than those required for anti-infective activity against common pathogens, for examples as determined by the minimum inhibitory concentrations required for 50% (MIC₅₀) or 90% (MIC₉₀) anti-bacterial activity. Without being limited to a particular theory, it is believed that macrolides exert anti-inflammatory effects independently of their anti-infective activity, which is based on binding to bacterial ribosomes and disrupting bacterial protein synthesis. Inflammatory lung conditions can thus be treated with lower doses and less frequent dosing than pulmonary infections, minimizing undesirable side effects that limit the usefulness of macrolides in many patients. This is particularly advantageous in treating chronic inflammation in the lungs, which often entails long-term dosing regimens (e.g., months to years). The instant methods further minimize adverse side effects by administering macrolides directly to sites of inflammation in the lungs, thereby maximizing local concentrations of the drug while minimizing systemic levels.

Accordingly, in some aspects, methods are provided comprising administering a “low dose” of an anti-inflammatory macrolide, wherein the low dose is below about 50 mg, or preferably below about 40 mg, or more preferably below about 30 mg of the anti-inflammatory macrolide. In some aspects, a low dose of an anti-inflammatory macrolide is administered by a nebulizer with an average deposition efficiency in the lungs of at least about 45%, or preferably at least about 55%, or more preferably at least about 65%, or even more preferably at least about 75% of the administered dose.

In further aspects, the use of a low dose allows aerosolized administration of an effective amount of an anti-inflammatory macrolide using a nebulizer in a significantly shorter dosing period than would be indicated with conventional dosing. For example, in some aspects, an effective amount of an anti-inflammatory macrolide is administered in less than about 10 minutes, or preferably less than about 7 minutes, or more preferably less than about 5 minutes using a nebulizer. Advantageously, the shorter dosing period required under the instant methods reduces occurrence of lung irritation and patient discomfort, and increases patient compliance. In addition, the instant methods allow treatment of inflammatory conditions at significantly lower cost than conventional methods.

In further aspects, the effective dose of anti-inflammatory macrolide is greater than 50 mg. For example, in some aspects, at least 50 mg of an anti-inflammatory macrolide is used to treat a chronic pulmonary infection and is administered for at least 2 weeks, or preferably at least one month, or more preferably at least 3 months, or even more preferably at least 6 months or longer. In some aspects, at least 50 mg of an anti-inflammatory macrolide is administered to a subject suffering from pulmonary inflammation that is not associated with a bacterial pulmonary infection. In some preferred aspects, the subject does not suffer from an infection with Streptococcus pneumoniae, Haemophilus influenzae, Staphylococcus aureus, Moraxella catarrhalis, Legionella pneumonia, Chlamydia pneumoniae, and/or Mycoplasma pneumoniae.

Certain methods provided herein are based on the discovery that some anti-inflammatory activities require higher local concentrations of macrolides, over more sustained durations, than are achievable using conventional dosing methods. Without being limited to a particular theory, it is believed that the anti-inflammatory effect of certain macrolides is based on multiple potential mechanisms of action at multiple potential therapeutic targets, and that these mechanisms and targets can also require different effective local concentrations of the macrolide. For example, some anti-inflammatory macrolides described herein exert a therapeutic effect by decreasing levels of inflammatory cells, such as alveolar macrophages (AMs) and neutrophils (PMNs), in the lungs, as well as by modulating levels of inflammatory and anti-inflammatory cytokines, such as IL-1α, IL-1β, IL-2, IL-4, IL-6, IL-10 and TNF-α. For example, in some instances, levels of IL-1α, IL-1β, IL-4, and/or TNF-α can be modulated at relatively lower macrolide concentrations while modulation of IL-2, IL-6 and/or IL-10 levels can require higher and/or more sustained macrolide concentrations. In some aspects, administering conventional doses of anti-inflammatory macrolides orally results in local concentrations of the macrolides in the lungs that are substantially lower than required for optimal efficacy during a portion of, or throughout the dosing interval.

Advantageously, the aerosolized administration of an anti-inflammatory macrolide according to methods provided herein achieves higher local concentrations of the macrolide in the lungs, and/or more sustained levels of the macrolide throughout the dosing interval, than would be achievable by oral administration of a comparable dose. Accordingly, in some aspects, the instant methods comprise administering an anti-inflammatory macrolide at substantially lower doses and/or with less frequent dosing than would be required to achieve a comparable effect with oral dosing. In further aspects, administering an anti-inflammatory macrolide according to the instant methods provides anti-inflammatory effects that cannot be achieved by orally administering the macrolide at any non-toxic dose.

In some aspects, administering an aerosolized dose of an anti-inflammatory macrolide according to methods provided herein results in a significantly higher local concentration of the macrolide at or in close proximity to a tissue, cell type, and/or region of the lungs at which the macrolide can exert an anti-inflammatory effect. For example, inflammatory mediators, such as chemokines, cytokines, NO, and the like, can be released at and act on several sites, including, e.g., alveolar macrophages (AMs), neutrophils, and/or respiratory (bronchiole and alveolar) epithelium. Without being limited by a particular theory, it is believed that orally administered macrolides preferentially accumulate in AMs, neutrophils, and other inflammatory cells, which carry the macrolide to sites of inflammation in the lungs. Thus, while oral administration of macrolides may achieve high drug concentrations in the lungs as a whole, the distribution of drug within the lungs is often limited to certain cells and/or regions. Advantageously, aerosolized administration of macrolides according to methods provided herein results in a wider distribution of the drug in the lungs and/or higher local concentrations at or near certain therapeutic targets than is achievable by oral administration. For example, in some aspects, methods provided herein result in substantially higher and/or more sustained macrolide concentrations within or near the respiratory epithelium than methods involving oral dosing. In some preferred aspects, the ability to exert an anti-inflammatory effect against a broader range of cells, tissues and other targets upon aerosol administration results in a substantially greater anti-inflammatory effect than seen with known oral dosing methods.

The term “pharmaceutical composition” refers to any composition that contains at least one therapeutically active agent and is suitable for administration to a subject. Pharmaceutical compositions suitable for aerosolized delivery of a therapeutic to the respiratory airways can be prepared by well-known and accepted methods in the art. See, for example, Remington: The Science and Practice of Pharmacy, 20th edition (ed. AR Gennaro), Mack Publishing Co., Easton, Pa., 2000.

A major challenge of aerosolized drug delivery is efficiently and evenly delivering effective doses of the drug of interest throughout the lungs, including the lower respiratory tract. Nebulizers are often preferred for delivering aerosolized drugs to the lungs, since they are capable of delivering the doses required for coating the large surface area of airway epithelium in the lungs. Thus, in some preferred aspects, the formulation, delivery device and/or protocol for administering an aerosolized anti-inflammatory macrolide are selected so as to allow deposition of an effective amount of the macrolide on surfaces throughout the lungs, including the peripheral regions of the lungs. Advantageously, the majority of the aerosol particles administered according to methods provided herein are distributed throughout the airways by the inhaled air stream and are deposited on physiological surfaces as a result of random diffusion within the air stream.

In further embodiments, the formulation, delivery device and/or protocol are selected to maximize local concentrations of the macrolide in the lungs relative to levels in plasma or peripheral organs (e.g., through targeted deposition to certain regions of the lung). For example, in certain aspects, the aerosolized macrolide is administered to the lungs with minimal systemic absorption through alveolar absorption and/or minimal deposition (e.g., through impaction) in the nasal passages, throat, and upper airways (and subsequent absorption from the GI tract).

In some aspects, a low dose of an aerosolized anti-inflammatory macrolide is administered to the lungs using a dry powder inhaler (DPI) in conjunction with a formulation suitable for dry power aerosolization.

In some preferred aspects, anti-inflammatory macrolides described herein are aerosolized and administered using a jet nebulizer, an ultrasonic nebulizer, or a vibrating membrane nebulizer.

Nebulizers are broadly known to those of skill in the art and the compositions, methods and formulations described herein are not limited to any specific type of nebulizer. Examples of suitable nebulizers and/or delivery devices and methods of their use for intrapulmonary administration are described in: U.S. Pat. Nos. 7,036,500; 7,029,656; 7,013,894; 6,994,083; 6,962,151; 6,929,003; 6,854,662; 6,748,945; 6,732,731; 6,729,327; 6,598,602; 5,853,002; 5,549,102; 5,435,282; 5,036,840; 7,077,126; 7,059,320; 6,983,747; 6,679,251; 6,606,990; 6,514,177; 513,727; 6,513,519; 6,464,388; 6,176,237; 6,085,741; 6,000,394; 5,957,389; 5,740,966; 5,596,982; 5,461,695; 5,458,136; 5,312,046; 5,309,900; 5,280,784; U.S. Patent Publication Nos.: 20060102172; 20060065267; 20060054166; 20060048772; 20060011196; 20050224076; 20050056274; 20050039741; 20040250816; 20030037788; 20030037785; 20020005196 and 20010054421 and the like, which are hereby incorporated by reference.

In describing the aerodynamic size distribution and/or particle size distribution of a formulation, the mass median aerodynamic diameter (“MMAD”) is the value wherein fifty percent of the particles by weight are smaller than the MMAD and 50% of the particles are larger. The geometric standard deviation (“GSD”) is a dimensionless number equal to the ratio between the MMAD and either 84% or 16% of the diameter size distribution (e.g., MMAD=2 m; 84%=4 m; GSD=4/2=2.0). The MMAD and GSD, together, can be used to describe the particle size distribution of an aerosol statistically, based on the weight and size of the particles. Suitable methods and devices for measuring aerodynamic size distributions are well known in the art, such as a multi-stage liquid impinger (MSLI).

Factors important for the localized administration of an aerosolized anti-inflammatory macrolide to the lungs by inhalation while minimizing systemic absorption are described, e.g., in Brand et al., Eur. Resp. J., 22(2): 263-7 (2003). For example, in various aspects, anti-inflammatory macrolides provided herein are administered, preferably using a nebulizer, such that: i) the aerosol particles have a MMAD predominantly between about 1-5 μm, and preferably between about 2-5 μm, wherein predominantly means at least 70%, preferably at least 80%, and more preferably at least 90% of all generated aerosol particles are within the stated size range; ii) the aerosol is inhaled with a flow rate of between about 200-500 ml/sec; iii) the effective amount of the macrolide is administered in an inhaled volume between about 1000-2000 ml; and 4) the aerosol is delivered by a nebulizer that allows for individualized determination and control of breathing volume and/or rate, and adjustment of flow rate, inhalation volume, and/or other variables accordingly.

In some aspects, the nebulizer is modified with a one-way flow valve which is breath-actuated and restricts delivery to the inhalation phase of the breath cycle. In some preferred aspects, a nebulizer and control device collectively known as the AKITA™ or AKITA²™ delivery system (Activaero GmbH, Germany) is utilized. The AKITA™ system incorporates a nebulizer such as the Pari LC Plus™ nebulizer (Pari Respiratory Equipment, Inc., Richmond, Va.) whereas the AKITA²™ system uses an aerosol generated from a modified Pari e-Flow nebulizer which uses a porous membrane driven by a piezoelectric oscillator. In some aspects, the nebulizer is capable of generating, storing and displaying individualized instructions for administering an anti-inflammatory macrolide to a targeted region of the lungs and/or controlling the rate, volume, particle distribution and/or other aspects of aerosol generation and delivery.

In some preferred aspects, at least about 45%, preferably at least about 55%, and more preferably at least about 65% of the anti-inflammatory macrolide is deposited in the peripheral regions of the lungs. In further aspects, less than about 10%, preferably less than about 5%, and more preferably less than about 3% of the anti-inflammatory macrolide is absorbed into the systemic circulation. In yet further aspects, at least about 65%, preferably at least about 75%, and more preferably at least about 85% of the loaded dose is administered as an aerosol for inhalation by the subject.

In various aspects, formulations of an anti-inflammatory macrolide provided herein have one or more of the following properties: a surface tension between about 10 to 70 dynes/cm, preferably between about 20 to 60 dynes/cm, and more preferably between about 30 to 50 dynes/cm; an osmolality between about 100 mOsm/kg to 500 mOsm/kg, and preferably between about 130 mOsm/kg to 400 mOsm/kg; a NaCl equivalency (% physiological) between about 0.15% NaCl and 0.35% NaCl, and preferably between about 0.2% NaCl and 0.3% NaCl; and a pH between about 5.0 and 7.0, or as otherwise indicated for stability of the anti-inflammatory macrolide.

Pharmaceutical excipients useful as carriers include stabilizers, bulking agents, pH adjusters or buffers, salts, and the like. Carriers can be in a crystalline or amorphous form, or a mixture thereof. Suitable bulking agents include compatible carbohydrates, polypeptides, amino acids or combinations thereof, such as aspartame, alanine, glycine, and the like. Suitable carbohydrates include monosaccharides, such as galactose, D-mannose, sorbose, and the like; disaccharides, such as lactose, trehalose, and the like; cyclodextrins, such as 2-hydroxypropyl-β-cyclodextrin; polysaccharides, such as raffinose, maltodextrins, dextrans, and the like; and alditols, such as mannitol, xylitol, and the like.

Also provided herein are pharmaceutical compositions comprising an anti-inflammatory macrolide encapsulated in a liposomal carrier, where such compositions are suitable for aerosolized administration to the lungs. Advantageously, the aerosolized administration of liposomal compositions described herein facilitates deposition of anti-inflammatory macrolides on surfaces throughout the lungs and a sustained release and absorption of such macrolides into surrounding tissues, resulting in uniform and sustained therapeutic levels of the macrolide in lung tissues with minimal levels in external tissues or systemic circulation. Moreover, in some aspects, the administration of liposomal compositions described herein facilitates targeting of anti-inflammatory macrolides to inflammatory cells within the lungs, including but not limited to, alveolar macrophages (AMs) and polymorphonuclear leukocytes (PMNs).

Methods for making and administering liposomal preparations optimized for aerosolized delivery to the lungs are known in the art and described, e.g., in U.S. Pat. Nos. 5,049,388 and 5,958,378, and U.S. Pat. Pub. No. 2005/0244339, each of which are incorporated by reference herein. In some preferred aspects, liposomal compositions described herein are “small particle aerosol” compositions, which comprise a colloid system with an air or oxygen-enriched air continuous phase, wherein the majority of aerosol particles or droplets are from about 1 to 5 microns in diameter with an aerodynamic mass median diameter ranging from about 1 to 3 microns and the majority of liposome-drug particles are less than about 1 micron in diameter. The smaller size of the liposome-drug particles relative to the aerosol particles allows multiple liposome-drug particles to reside in each aerosol particle, providing a uniform distribution of drug in the aerosolized composition. Advantageously, such small particle aerosol compositions can be administered by a nebulizer to provide high concentration on the respiratory epithelium and a steady rate of absorption into the circulation, without the hazard of peak levels that can be associated with large oral or intravenous doses of drug. Accordingly, in some aspects, administering small particle aerosol compositions according to methods described herein delivers high doses of an anti-inflammatory macrolide to the epithelium of the respiratory tract and achieves local concentrations unachievable by other formulations and routes of administration.

Lipids suitable for making compositions provided herein include phospholipids, such as natural lecithins derived from egg-yolk or soya-bean, sphingomyelin derived from beef brain, synthetic lecithins (e.g., dimyristoyl-phosphatidylcholine, dipalmitoylphosphatidylcholine or distearoyl-phosphatidylcholine), or unsaturated synthetic lecithins (e.g., dioleylphosphatidylcholine or dilinoleyl-phosphatidylcholine). A single phospholipid or a mixture of phospholipids can be used. Sterols, such as cholesterol or ergosterol, can be added to increase stability of the liposomal bilayers, and lipids possessing a positive or negative change, such as phosphatidylethanolamine, beef brain ganglioside or phosphatic acid can be used to render an appropriate charge to liposomes and to increase the size of aqueous compartments. Mixtures of lipids can be used to render the liposomes more fluid or rigid and to increase or decrease permeability.

Liposomes can be prepared by a variety of methods, which generally involve dispersing a phospholipid or mixture of lipids into a suitable container; removing the organic solvent from the lipid mixture, for example by evaporation, rotary evaporation under vacuum or lyophilization, to form a dry film; and dispersing the lipid film in an aqueous medium, such as distilled water, isotonic saline or a buffered solution, to form liposomes. For example, in one method, phosphatidylcholine is dissolved in re-distilled t-butanol, the solution is frozen and the solvent is removed under vacuum using a commercial freeze-dryer. Sterile pyrogen-free distilled water is added to the freeze dried powder and the bottle shaken to disperse the powder. The resulting milky suspension can be examined microscopically to confirm formation of liposomes that are heterogeneous in size ranging from less than about 1 micron up to about 10 microns.

An anti-inflammatory macrolide and/or other active agent can be incorporated into liposomes using a variety of methods. Whether the macrolide or other agent is associated with the lipid portion of the liposomes or resides in the aqueous compartments depends on the physical and chemical properties of the compound. Two general methods of incorporating active agents into liposomes are described below. The first method is useful for incorporating lipid soluble or lipid-bound compounds into liposomes. Briefly, egg lecithin (phosphatidylcholine) or a similar phospholipid is dissolved in an organic solvent and the lipid soluble compound of interest is added. The solution is frozen and the solvent is removed using a commercial freeze-dryer, and liposomes are formed by adding a suitable aqueous medium, such as isotonic saline, and vigorously shaking the container. Organic solvents, such as chloroform, n-butanol, t-butanol, and pyridine can be used as necessary to promote interaction of a lipid-soluble compound with the phospholipid.

The second method allows for incorporates active compounds into liposomes without regard to the solubility characteristics of the compound by derivatizing the compound with a lipid moiety which anchors the compound to the liposomal biolayer. Phosphatidylethanolamine and palmitic acid are useful lipid moieties, but a variety of lipids can be utilized as well. In an exemplary aspect, a compound is mixed with a lipid derivative, such as N-hydroxy-succinimide ester of palmitic acid, N-succinyl-phosphatidylethanolamine, or phosphatidylethanolamine, in the presence of a dehydrating agent, such as N—N,-dicyclohexylcarbodiimide, in a suitable solvent. The lipid derivative is then purified and incorporated into liposomes, for example by mixing the derivatized compound with egg lecithin or a similar phospholipid in an organic solvent, freezing the mixture, and then adding the frozen mixture to a suitable aqueous medium and vigorously shaking it.

In other aspects, unit dose formulations are provided comprising an anti-inflammatory macrolide in solution for aerosolized administration to the lungs by inhalation. In some aspects, the unit dose formulations are packaged with instructions for locally administering the aerosolized macrolide to a targeted region or regions of the lungs using a nebulizer or other device to achieve a substantially uniform distribution throughout the targeted region(s) with minimal systemic absorption.

In some aspects, the unit dose formulations comprise a container designed to hold and store volumes of the anti-inflammatory macrolide corresponding to a single unit doses, or multiple unit doses, including e.g., 2, 3, 4, or 5 unit doses. In some aspects, the unit dose formulations comprise a plastic ampoule filled with an anti-inflammatory macrolide and sealed under sterile conditions. Preferably, the unit dose ampoule is provided with a twist-off tab or other easy opening device for opening of the ampoule and delivery of the macrolide antibiotic formulation to an inhalation device. Ampoules for containing drug formulations are well known to those skilled in the art (see, for example, U.S. Pat. Nos. 5,409,125, 5,379,898, 5,213,860, 5,046,627, 4,995,519, 4,979,630, 4,951,822, 4,502,616 and 3,993,223, the disclosures of which are incorporated herein by this reference). In some aspects, the unit dose ampoules are capable of being inserted directly into an inhalation device, such as a nebulizer, for aerosolization of the anti-inflammatory macrolide and delivery to a subject by inhalation.

In some aspects, liquid unit dose formulations provided herein for aerosolized administration contain less than about 50 mg, less than from about 45 mg, or less than about 40 mg of an anti-inflammatory macrolide per dose. In some aspects, the liquid unit dose formulations contain less than about 25 mg, less than about 20 mg, or less than about 15 mg of an anti-inflammatory macrolide per dose. In some preferred aspects, the anti-inflammatory macrolide antibiotic is erythromycylamine.

In other aspects, liquid unit dose formulations provided herein for aerosolized administration contain between about 1 mg to about 100 mg, preferably between about 5 mg and about 75 mg, and more preferably between about 10 mg and about 50 mg of an anti-inflammatory macrolide. In some aspects, the anti-inflammatory macrolide is azithromycin, clarithromycin, or roxithromycin.

In some aspects, unit dose formulations provided herein for aerosolized administration contain less than about 5.0 ml, preferably less than about 3.0 ml and more preferably less than about 2.0 ml of a liquid anti-inflammatory macrolide. In further aspects, liquid unit dose formulations provided herein contain between about 1 to about 100 mg/ml, preferably between about 5 to about 50 mg/ml, and more preferably between about 10 and 25 mg/ml of an anti-inflammatory macrolide in a physiologically acceptable carrier. In some preferred aspects, small particle aerosol liposomal compositions provided herein contain about 1-50 mg/ml, preferably about 5-45 mg/ml, and more preferably about 10-40 mg/ml of an anti-inflammatory macrolide.

Also provided herein are kits comprising one or more liquid unit dose formulations of an anti-inflammatory macrolide and instructions for the localized administration of an aerosolized anti-inflammatory macrolide throughout the lungs, including, e.g., optimal flow rates, inhalation volumes, and particle size distributions for delivering the macrolide to a targeted region of the lungs. In some aspects, the kits further comprise equipment, such as nebulizer attachments or accessories, useful in administering liquid formulations provided herein.

In some aspects, methods and compositions provided herein include a second therapeutic agent for treating an inflammatory lung condition or a conditions or side effects associated with an inflammatory lung condition. Such agents include, for example, non-macrolide anti-inflammatory agents, such as glucocorticoids (beclomethasone, flunisolide, budesonide, triamcinolone, prednisolone, dexamethasone, or fluticasone); non-steroidal anti-inflammatory agents (e.g., ibuprofen, tacrolimus, cromolyn, nedocromil, refecoxib, or celecoxib); non-macrolide antimicrobial agents (e.g., amikacin, gentamicin, kanamycin, neomycin, netilmicin, paromomycin, streptomycin, or tobramycin); antihistamines (e.g., diphenhydramine, fexofenadine, cetirizine, or loratadine); cholinergic receptor antagonists (e.g., ipratropium bromide or tiotropium); neurokinin receptor antagonists; leukotriene receptor antagonists; decongestants; phosphodiesterase inhibitors; or beta-adrenergic receptor antagonists (albuterol, bitolterol, epinephrine, fenoterol, formoterol, isoetharine, isoproterenol, metaproterenol, pirbuterol, procaterol, racepinephrine, salmeterol, or terbutaline).

In some aspects, an anti-inflammatory macrolide described herein is administered with cyclosporine for the treatment of an inflammatory lung condition.

Having now generally described the invention, the same will be more readily understood through reference to the following examples which are provided by way of illustration, and are not intended to be limiting of the disclosed invention, unless specified.

### EXEMPLARY ASPECTS

### Example #1

The pharmacokinetics of erythromycylamine in lung and plasma was investigated following multiple administration of erythromycylamine via the endotracheal route in the rat.

A group of 33 male Sprague Dawley rats used in the study were dosed endotracheally at 5 mg/kg erythromycylamine sulfate (5 mg/kg free base, Advinus Therapeutics Pvt. Ltd.) in Milli-Q water (formulation strength: 3.33 mg/mL, dosing volume: 1.5 mL/kg) up to three times at 0, 48 and 96 h during the study. For dose administration, animals were lightly anesthetized with diethyl ether. Under anesthetic condition the mouth of the animal was opened with the help of tweezers, tongue was pulled out and the dose was administered into the tracheal lumen with the help of a blunt hypodermic needle.

Lung and plasma samples were collected at 0.033, 24 and 48 h time points from three rats at each time points each (total of 9 rats). The remaining 24 rats were administered a second dose on day 3 (48 h); lung and plasma samples were collected at 48, 72 and 96 h time points from three rats at each time point (total of 9 rats. The remaining 15 rats were administered a third dose on day 5 (96 h); lung and plasma samples were collected at 96, 120, 144, 192 and 240 h time points. Additionally, spot blood samples were collected at 1 hour post first-dose from all animals, except those used for the 2 min post dose samples, and analyzed as a control to assess whether the formulation was correctly delivered to all animals.

For sample collection, animals were subjected to ether anesthesia and blood samples were withdrawn by retro orbital plexus. Whole blood samples were collected on ice in a labeled micro centrifuge tube containing dipotassium EDTA as anticoagulant (20 μL of 200 mM K₂ EDTA solution per mL of blood). Plasma was separated by centrifuging the whole blood at about 2500 g for 10 minutes at 4° C. within 1 h of sample collection. Separated plasma was stored below −70° C. until bioanalysis.

Following collection, the lungs were washed three times in ice-cold phosphate buffered saline (PBS), gently blotted dry, weighed and placed in tared polypropylene tubes, and stored below −70° C. until bioanalysis. Plasma samples were separated by centrifuging whole blood and stored below −70° C. until bioanalysis.

A specific and sensitive LC-MS/MS method (lower limit of quantitation of 10.81 ng/mL) was developed and employed for quantification of erythromycylamine in plasma and lung samples. Lung samples were diluted 10-fold in buffer and homogenized prior to analysis. All The plasma and lung homogenates were extracted for erythromycylamine by liquid-liquid extraction with t-butyl methyl ether and analyzed using liquid chromatography and mass spectrometric method. Oleandomycin was used as internal standard.

The concentrations of erythromycylamine were expressed as ng/mL for plasma and μg/g for lung tissues. The obtained tissue concentrations in ng/mL were converted to μg/g tissue by multiplying by homogenate volume, and dividing by the tissue weight. The extravascular model in the Non-Compartmental-Analysis module of WinNonlin® Enterprise software (Version 5.1.1) was used to assess pharmacokinetic parameters. The overall exposure data are summarized in Table 1.

TABLE 1

| | Plasma | | Lung^{a} | |
|---|---|---|---|---|
| AUC | | Ratio | | Ratio |
| AUC₀₋₄₈ (ug · h/mL) | 130.02 | — | 4362.90 | — |
| AUC₄₈₋₉₆ (ug · h/mL) | 137.07 | 1.05 | 5664.25 | 1.30 |
| AUC₉₆₋₁₄₄ (ug · h/mL) | 145.91 | 1.06 | 5466.22 | 0.96 |
| AUC₀₋₂₄₀ (ug · h/mL) | 413.50 | — | 19718.60 | — |
| Cₘₐₓ | Plasma | | Lung^{a} | |
| C_{max, 0-48} (ug/mL) | 10.83 | — | 290.70 | — |
| C_{max, 48-96} (ug/mL) | 11.41 | 1.05 | 369.39 | 1.27 |
| C_{max, 96-144} (ug/mL) | 12.13 | 1.06 | 311.68 | 0.84 |

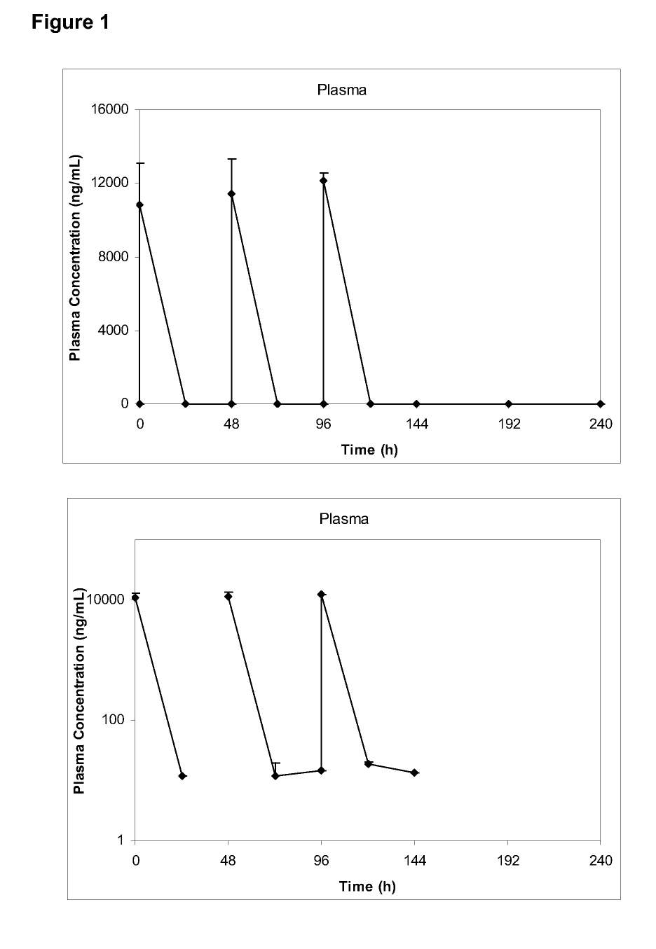

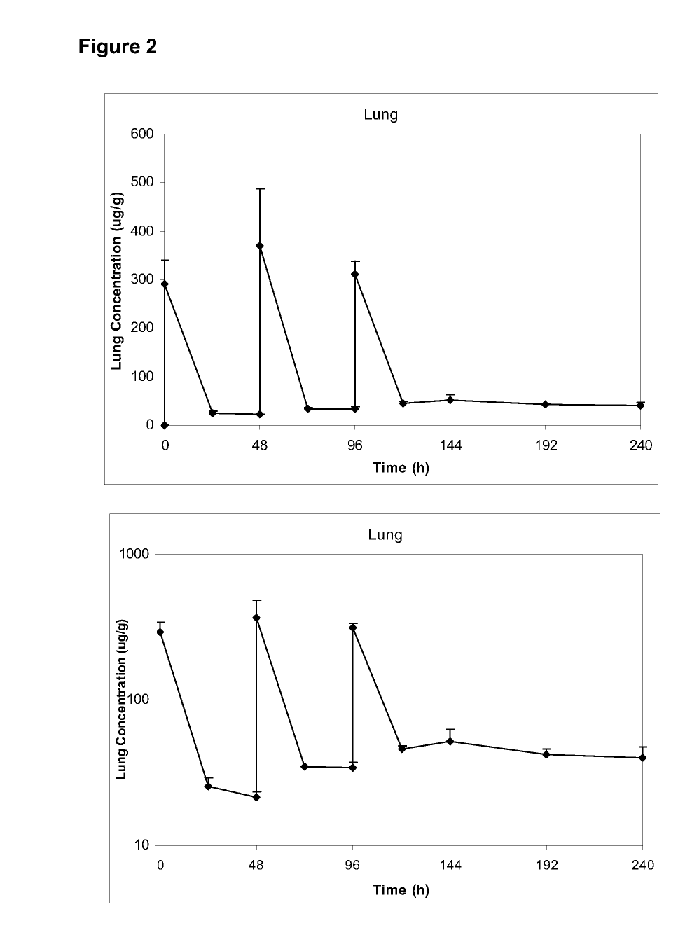

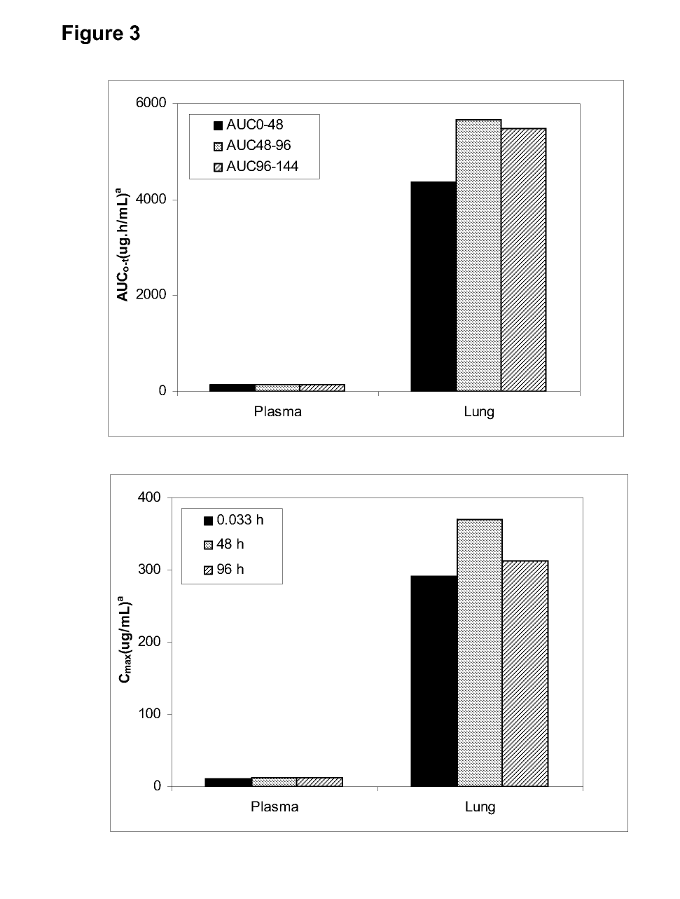

The mean plasma and lung concentrations of erythromycylamine at various time points following endotracheal administration are presented in Table 2 and the concentration vs. time profiles are shown in FIGS. 1-3. Pharmacokinetic parameters are presented in Table 3, and accumulation data is presented in Table 4.

TABLE 2

| Plasma Concentration | | | | Lung Concentration | | | |
|---|---|---|---|---|---|---|---|
| Time (h) | Conc. (ng/mL) | SD | % CV | Time (h) | Conc. (ug/g) | SD | % CV |
| 0.033 | 10827.22 | 2261.83 | 20.89 | 0.03 | 290.70 | 50.60 | 17.41 |
| 24 | 11.90^{a} | NA^{b} | NA | 24.00 | 25.69 | 3.45 | 13.42 |
| 48 | NA | NA | NA | 48.00 | 21.53 | 1.85 | 8.58 |
| 48.033 | 11409.98 | 1933.24 | 16.94 | 48.033 | 369.40 | 116.88 | 31.64 |
| 72 | 11.59^{c} | NA | NA | 72.00 | 34.60 | 0.48 | 1.39 |
| 96 | 14.29^{a} | NA | NA | 96.00 | 33.97 | 3.53 | 10.39 |
| 96.033 | 12134.27 | 416.57 | 3.43 | 96.033 | 311.68 | 25.97 | 8.33 |
| 120 | 18.53 | 2.27 | 12.26 | 120.00 | 46.19 | 2.52 | 5.46 |
| 144 | 13.55^{a} | NA | NA | 144.00 | 51.87 | 10.70 | 20.63 |
| 192 | NA | NA | NA | 192.00 | 41.93 | 3.63 | 8.65 |
| 240 | NA | NA | NA | 240.00 | 39.85 | 7.47 | 18.75 |

TABLE 3

| Parameters | Plasma | Parameters | Lung |
|---|---|---|---|
| Tₘₐₓ | 96.03 | Tₘₐₓ | 48.03 |
| (h) | | (h) | |
| Cₘₐₓ | 12.13 | Cₘₐₓ | 369.39 |
| (ug/mL) | | (ug/g) | |
| AUC₀₋₂₄₀ | 413.50 | AUC₀₋₂₄₀ | 19718.60 |
| (ug · h/mL) | | (ug · h/g) | |
| AUC₀₋₄₈ | 130.02 | AUC₀₋₄₈ | 4362.90 |
| (ug · h/mL) | | (ug · h/g) | |
| AUC₄₈₋₉₆ | 137.07 | AUC₄₈₋₉₆ | 5664.25 |
| (ug · h/mL) | | (ug · h/g) | |
| AUC₉₆₋₁₄₄ | 145.91 | AUC₉₆₋₁₄₄ | 5465.22 |
| (ug · h/mL) | | (ug · h/g) | |

TABLE 4

| | |
|---|---|
| Plasma | Lung^{a} |

| | | Ratio | | Ratio |
|---|---|---|---|---|
| AUC₀₋₄₈ (ug · h/mL) | 130.02 | — | 4362.90 | — |
| AUC₄₈₋₉₆ (ug · h/mL) | 137.07 | 1.05 | 5664.25 | 1.30 |
| AUC₉₆₋₁₄₄ (ug · h/mL) | 145.91 | 1.06 | 5466.22 | 0.96 |
| C_{max, 0-48} (ug/mL) | 10.83 | — | 290.70 | — |
| C_{max, 48-96} (ug/mL) | 11.41 | 1.05 | 369.39 | 1.27 |
| C_{max, 96-144} (ug/mL) | 12.13 | 1.06 | 311.68 | 0.84 |

After each dose erythromycylamine concentration was highest at the first measured time point (2 min. post dose) and then declined as a function of time. At the terminal phase, profiles flattened out after 120 hours. Lung concentrations of erythromycylamine at 240 hour were approximately 11% of peak concentration. Based on the plasma and lung exposures, erythromycylamine did not substantially accumulate in lung or plasma following multiple endotracheal administrations at a dosing interval of 24 h. The peak whole lung concentration (Cmax) and overall exposure (AUC₀₋ₗₐₛₜ) of erythromycylamine were 369.39 μg/g and 19718.60 μg·h/g, respectively.

The peak plasma concentration (Cₘₐₓ) and overall exposure (AUC₀₋ₗₐₛₜ) of erythromycylamine were 12.13 μg/mL and 413.50 μg·h/mL, respectively. Plasma concentrations of erythromycylamine were quantifiable in all the post dose samples at 0.033 h. The appearance of concentrations in micrograms in the first sampled time point (0.033 h) suggest that erythromycylamine is absorbed from lung. As expected, concentrations in plasma samples were significantly lower (98%) than those in corresponding lung samples. Erythromycylamine concentrations determined in the spot plasma samples (1 hour post dose) collected from all animals suggested that the endotracheal dosing was 100% successful.

In conclusion, the results of this study suggest that the exposure of erythromycylamine in lung is significantly higher than the plasma exposure with no substantial accumulation following multiple endotracheal administration of erythromycylamine at dose of 5 mg/kg.

### Example #2

The pharmacokinetics of erythromycylamine in lung and plasma were investigated following endotracheal administration of erythromycylamine in the rat.

A group of sixty male Sprague Dawley rats were used in the study. The animals were dosed endotracheally at 5 mg/kg erythromycylamine sulfate (5 mg/kg free base, Advinus Therapeutics Pvt. Ltd.) in water (formulation strength: 3.33 mg/mL, dosing volume: 1.5 mL/kg). The lung and blood samples were collected from five rats at 0.5, 1, 2, 4, 8, 24, 48, 72, 96, 120, 144 and 168 h post-dose (N=5 at each time point). Additionally, spot blood samples were collected from all animals at 1 hour post dose and analyzed as a control to assess whether the formulation was correctly delivered to all animals.

For sample collection, animals were subjected to ether anesthesia and blood samples were withdrawn by retro orbital plexus. Whole blood samples were collected on ice in a labeled micro centrifuge tube containing dipotassium EDTA as anticoagulant (20 uL of 200 mM K₂ EDTA solution per mL of blood). Plasma was separated by centrifuging the whole blood at about 2500 g for 10 minutes at 4° C. within 1 h of sample collection. Separated plasma was stored below −70° C. until bioanalysis.

After blood collection, animals were euthanized by exsanguination under deep ether anesthesia. The individual lobes of the lung (left lobe (LL), accessory lobe (AL), right middle lobe (RML), right cranial lobe (RCrL) and right caudal lobe (RCaL)) were dissected out.

Following collection, the lungs were washed three times in ice-cold phosphate buffered saline (PBS), sectioned into respective lobes, gently blotted dry, weighed and placed in tared polypropylene tubes, and stored below −70° C. until bioanalysis. Plasma samples were separated by centrifuging whole blood and stored below −70° C. until bioanalysis.

A specific and sensitive LC-MS/MS method (lower limit of quantitation of 10.65 ng/mL) was employed for quantification of erythromycylamine in the plasma and lung samples. The plasma and tissue homogenates were extracted for erythromycylamine by liquid-liquid extraction with t-butyl methyl ether and analyzed using liquid chromatography and mass spectrometric method. Lung samples were diluted 10-fold in buffer and homogenized prior to analysis. Oleandomycin was used as internal standard.

The concentrations of erythromycylamine were expressed as ng/mL for plasma and μg/g for lung tissues. The obtained tissue concentrations in ng/mL were converted to μg/g tissue by multiplying by homogenate volume, and dividing by the tissue weight. The average lung concentration of erythromycylamine was determined by taking the sum of the erythromycylamine amounts for each individual lobe at each time point, and dividing by the whole lung weight. The extravascular model in the Non-Compartmental-Analysis module of WinNonlin® Enterprise software (Version 5.1.1) was used to assess pharmacokinetic parameters including AUC and Cₘₐₓ.

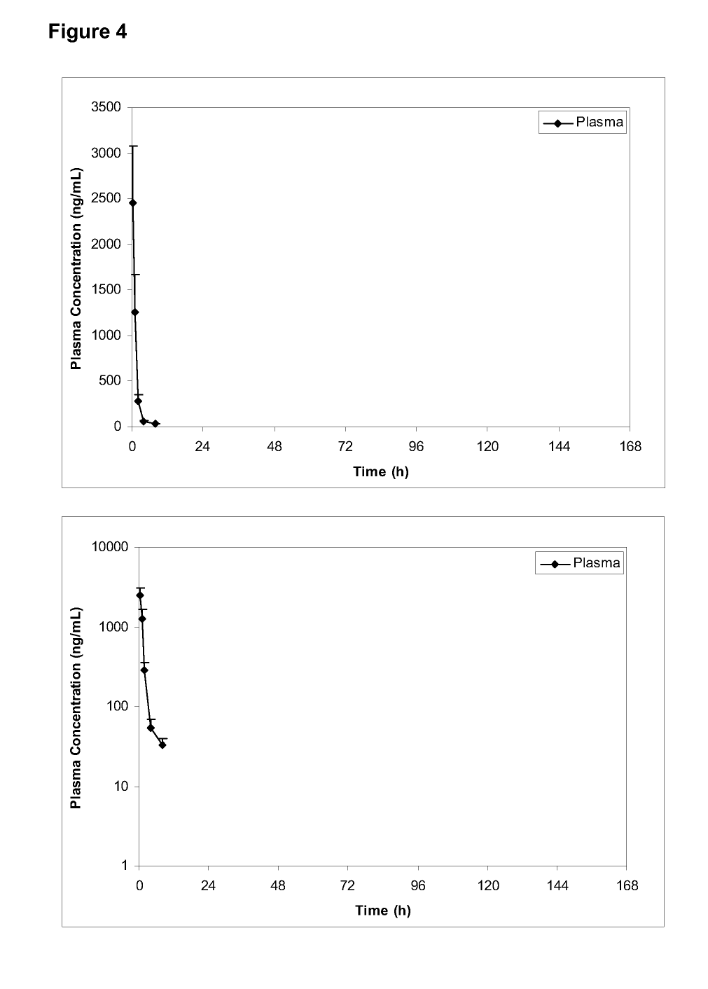

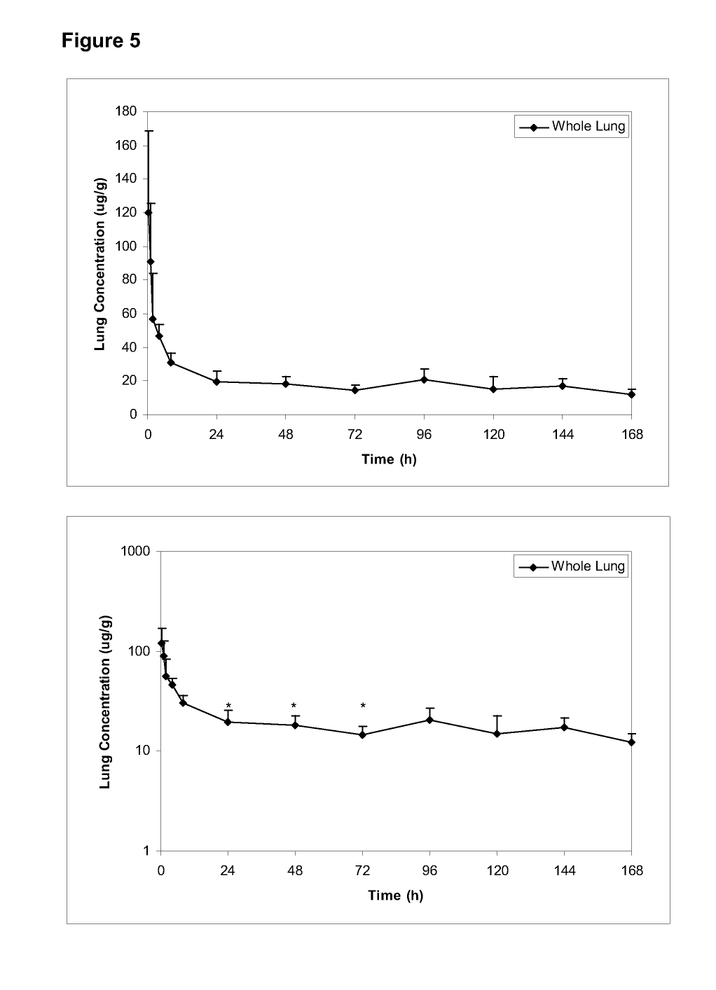

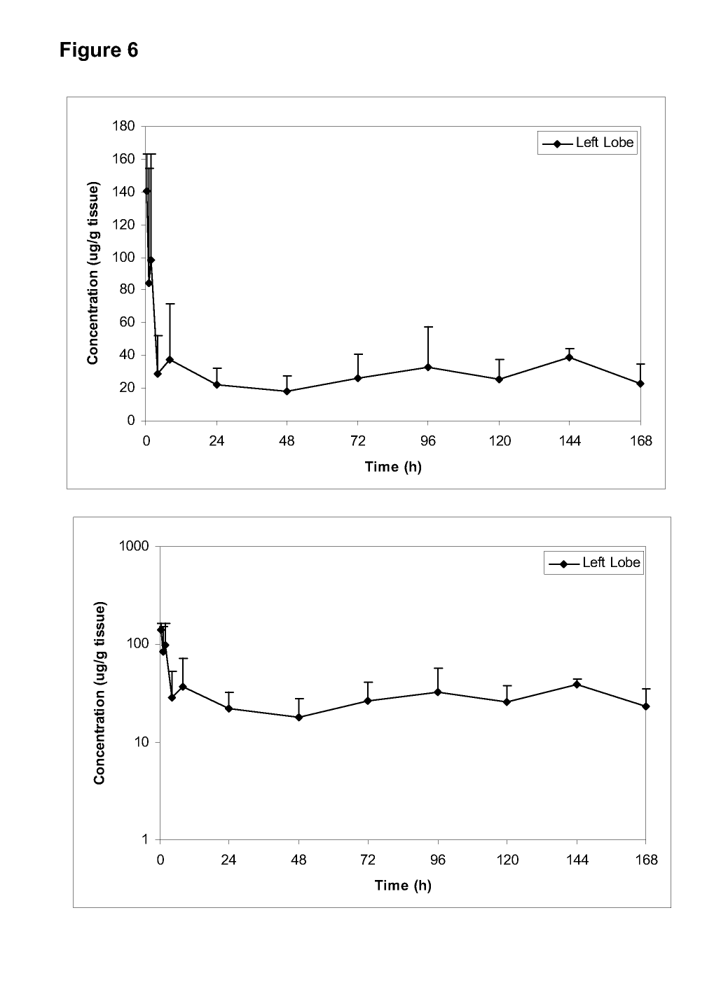

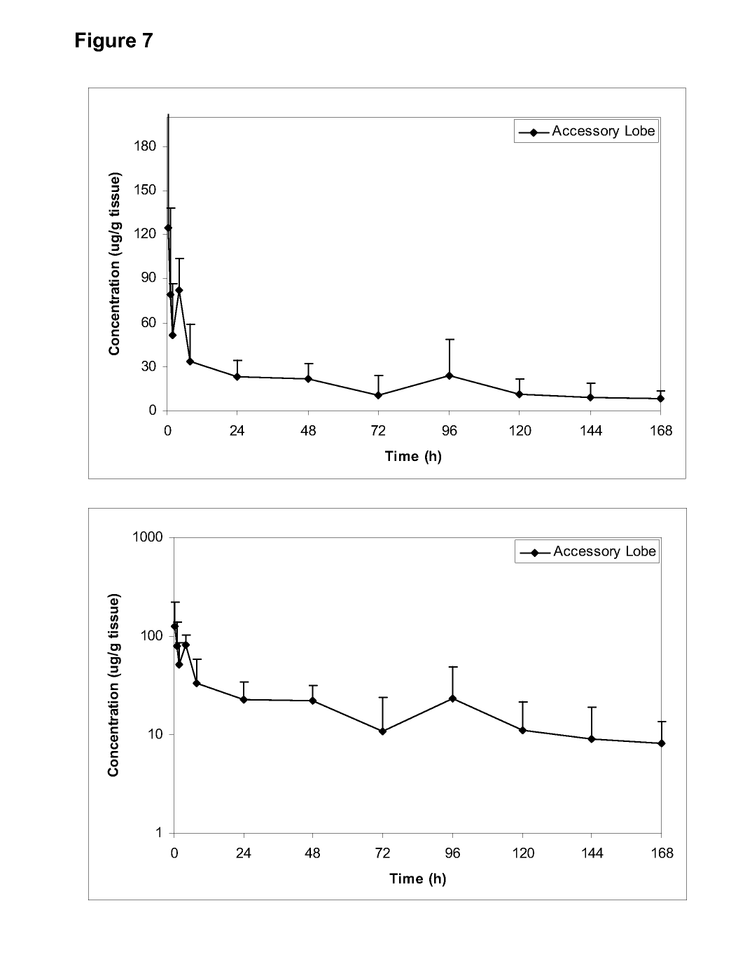

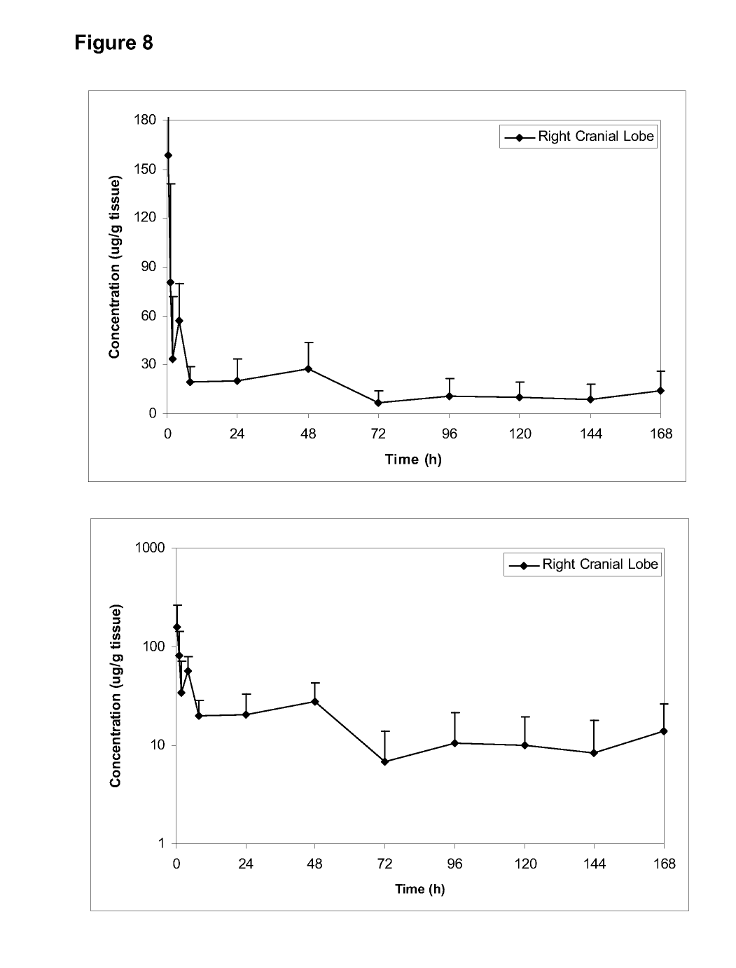

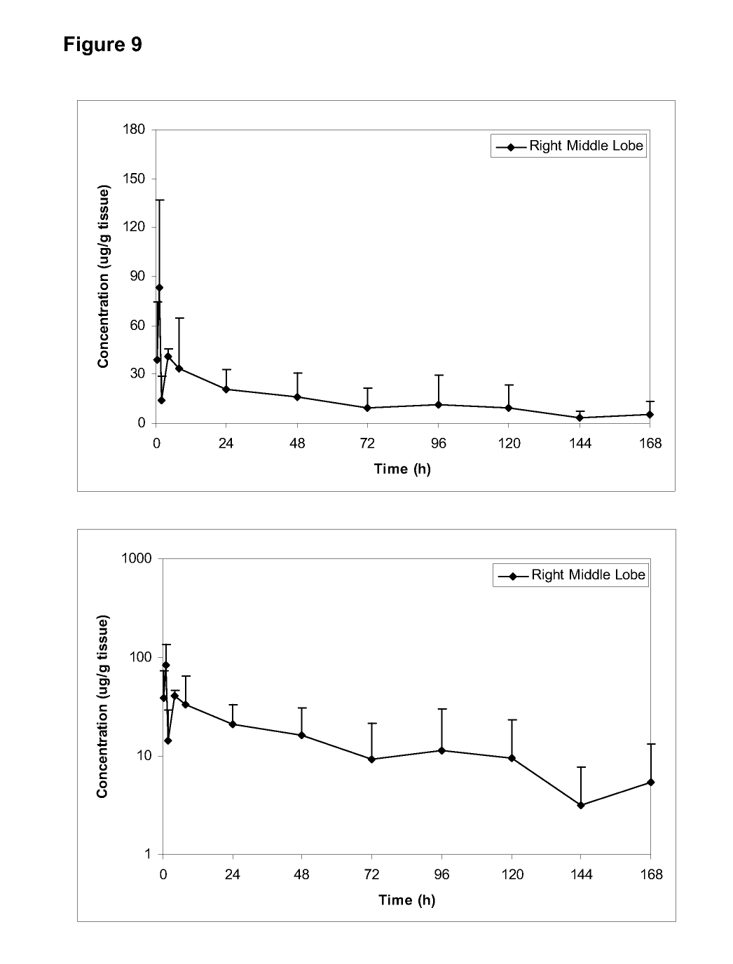

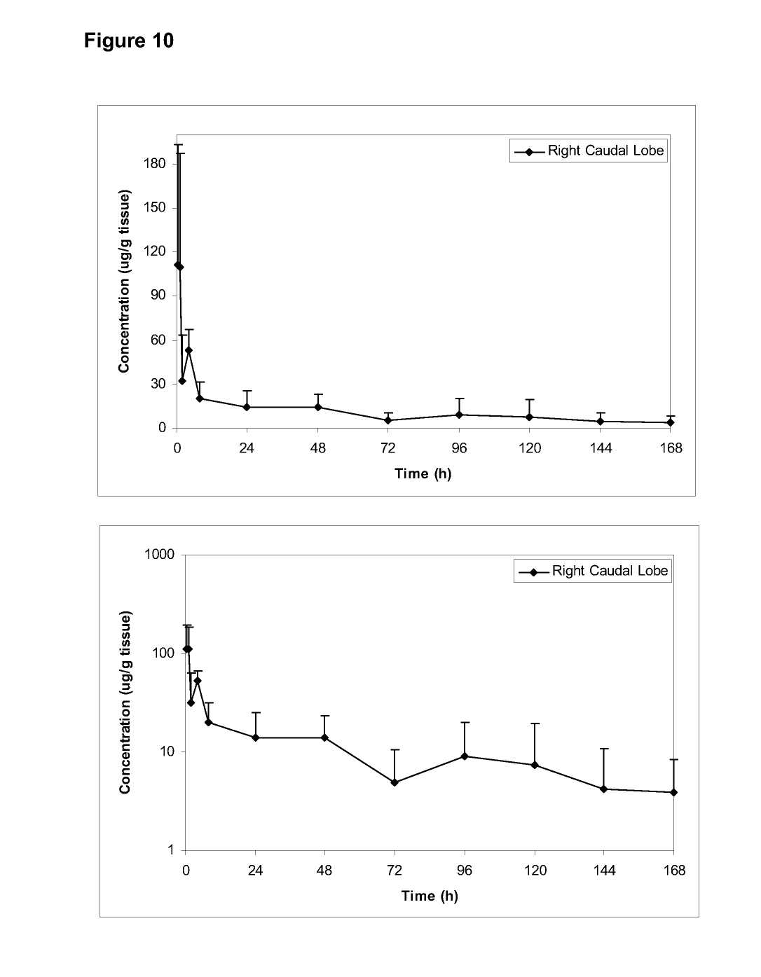

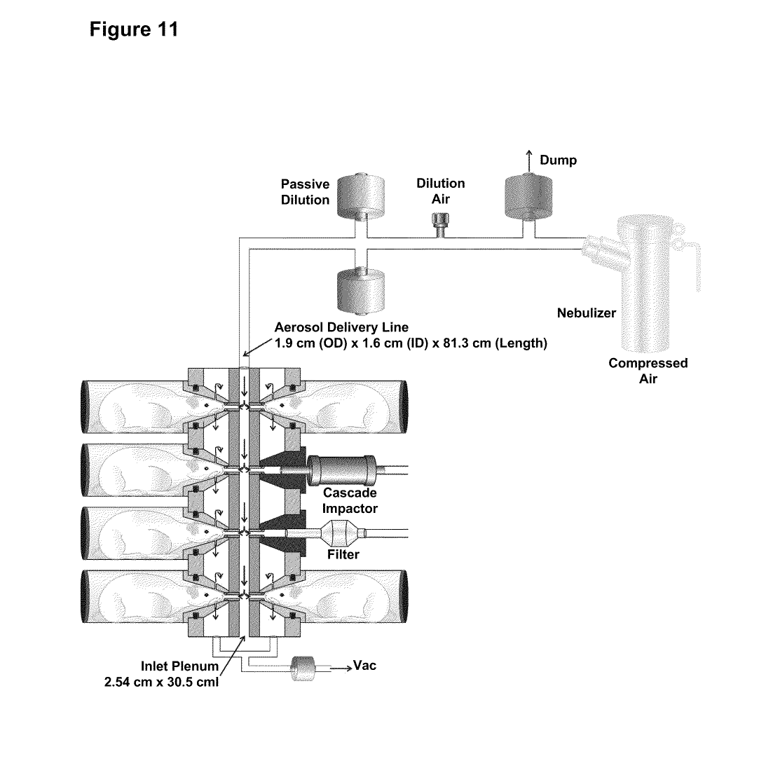

The plasma concentration of erythromycylamine vs. time is presented in FIG. 4 and whole lung erythromycylamine concentrations vs. time are shown in FIG. 5. Erythromycylamine concentrations in separate regions (lobes) of the lung vs. time are shown in FIGS. 6 through 10. Summary pharmacokinetic parameters are presented in Table 5.

TABLE 5

| | Tₘₐₓ | Cₘₐₓ | AUC₀₋ₗₐₛₜ | T_{1/2} |
|---|---|---|---|---|
| Matrix | (h) | (ng/mL) | (ng · h/mL) | (h) |
| Plasma | 0.50 | 2453 | 2823 | 1.2 |
| Whole lung | 0.50 | 120 | 3264 | 114.6 |

Following endotracheal dosing, the mean peak erythromycylamine concentration (Cₘₐₓ) in whole lung was 120 μg/g tissue, and the overall whole lung exposure (AUC₀₋ₗₐₛₜ) was 3264 μg·h/g tissue. Concentrations of erythromycylamine at 168 hour were approximately 10% of peak lung concentration. Whole lung concentrations of erythromycylamine were highest at the first measured time point (0.5 h post dose) and then declined with time for up to 24 hours; the profiles flattened out thereafter (FIG. 5). Similar declining profiles were seen in the individual lung lobes as well (FIGS. 6 to 10). The terminal part of the curve from 48 h to 168 h was relatively flat; a conservative selection of the terminal time points for the T_{1/2} assessment was made, and the T_{1/2} in lung was estimated to be approximately 115 hour.

Erythromycylamine was quantifiable in plasma from 0.5 to 8 hour after endotracheal dosing into the lung. The peak plasma concentration (Cₘₐₓ) and overall exposure (AUC₀₋ₗₐₛₜ) of erythromycylamine were 2.45 μg/mL and 2.82 μg·h/mL, respectively, and the plasma half-life was approximately 1.2 hour. As expected, concentrations in plasma samples (expressed as ng/mL) were significantly lower than those in corresponding lung samples (expressed as μg/g).

Erythromycylamine concentrations were determined in the spot plasma samples (1 hour post dose) collected from all animals to assess reproducibility of the dosing technique. The results suggested that the endotracheal dosing was successful in 58/60 animals. Two replacement rats were dosed subsequently, and correctness of dosing was similarly assessed from the concentration at 1 hour time point. Reproducibility of the dosing was further supported by the mean concentration-time profiles in the individual lung lobes, which all showed a declining trend with time (FIGS. 6 to 10). Further, erythromycylamine concentrations in the different lung lobes were similar at each time point, indicating that the formulation was distributed uniformly in the lung after endotracheal administration. An exception was the 0.5 h and 2 h samples of the right middle lobe, where concentrations were slightly lower than in other regions.

In conclusion, the pharmacokinetics of erythromycylamine in the lungs were investigated after endotracheal dosing. The data suggest that erythromycylamine has a long half-life in lung tissue in the rat (approximately 115 hour).

### Example #3

The effectiveness of aerosolized erythromycylamine in inhibiting inflammation of the lung was investigated by measuring neutrophil infiltration in the bronchoalveolar lavage fluid and cytokine profiles in lung tissues of a F344 Rat LPS inflammation model. These results were benchmarked against a previously characterized positive control, budesonide (IT), to ensure performance of the model. Erythromycylamine content in lung was also measured to evaluate pharmacokinetics/pharmacodynamics of the aerosolized drug.

Table 6 shows the experimental design. Four groups of rats were exposed to erythromycylamine sulfate aerosols (target doses of 1 and 5 mg/kg) approximately 48 and 12 hr prior to a 10 minute lipopolysaccharides (LPS) inhalation exposure. Sixteen (16) positive control animals, Groups C and D, were intratracheally instilled with budesonide sulfate (1.5 mg/kg) approximately 60 minutes (±15 minutes) prior to LPS exposure.

TABLE 6

| | | | Dose | Exposure | Exposure | Next Post LPS |
|---|---|---|---|---|---|---|
| Group | Test Article | N | (mg/kg) | method | (h) | Exposure (h) |
| A | control | 10 | — | — | — | 4 |
| B | control | 10 | — | — | — | 24 |
| C | budesonide | 10 | 1.5 | IT | −4 | 4 |
| D | budesonide | 10 | 1.5 | IT | −4 | 24 |
| E | erythromycylamine | 10 | 1 | inhalation | −48, −12 | 4 |
| F | erythromycylamine | 10 | 1 | inhalation | −48, −12 | 24 |
| G | erythromycylamine | 10 | 5 | inhalation | −48, −12 | 4 |
| H | erythromycylamine | 10 | 5 | inhalation | −48, −12 | 24 |

Animals were exposed to erythromycylamine aerosols per the protocol for 15 or 60 minutes. Oxygen (%) and temperature (° C.) were monitored and recorded throughout the exposure periods and ranged from 19.3% to 20.2% and 22.1° C. to 24.8° C. Average aerosol concentrations over the 2 exposures were 0.565 (0.110) mg/L and 0.489 (0.049) mg/L. Particle size distribution, which is characterized by mass median aerodynamic diameter (MMAD) and geometric standard deviation (GSD), was measured utilizing a mercer style impactor (In-Tox Products, Inc.) operated at approximately 2 liters per minute.

Budesonide sulfate was formulated at 1 mg/mL and animals received 270-325 μL, depending on pre-study body weight. Control animals were not exposed or administered any material. All rats were exposed once to LPS by nose-only inhalation at a 5 mg/m³ aerosol concentration. Particle size distribution was not measured during the LPS exposures.

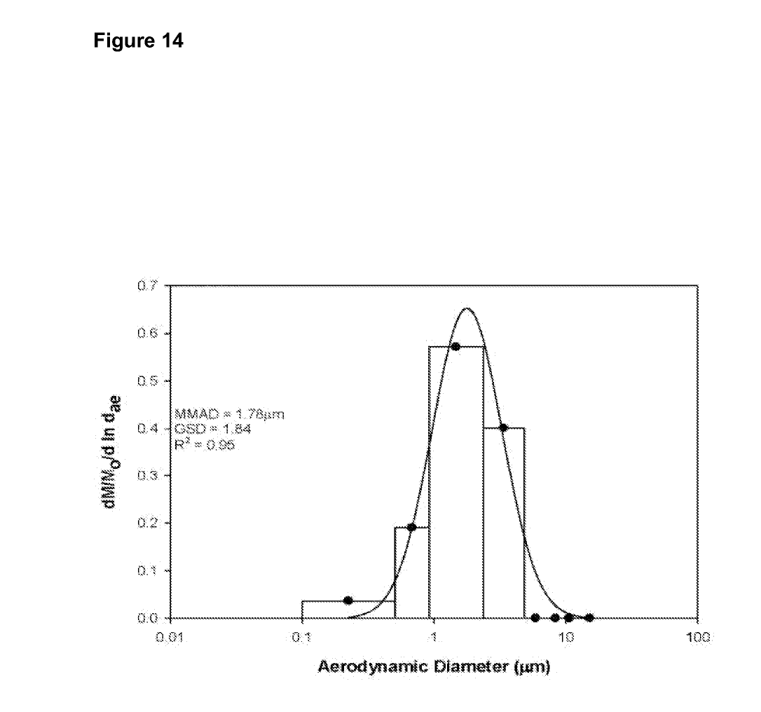

The actual concentrations of erythromycylamine in the exposure atmospheres were determined by gravimetric analysis of timed filter samples collected directly from a nose-only exposure port during each of the exposure. In addition, one impactor sample was obtained per test article to verify particle size distribution (PSD) during the exposures, which was 1.78 μm MMAD and 1.84 GSD (FIG. 14). Dose was estimated for the two exposure groups using an average body weight of 200 g, exposure periods of 15 and 60 minutes, and an estimated deposition fraction of 10%. Estimates were within 20% of target doses (0.54 and 2.15 mg/kg, subsequently referred to as 0.5 and 2.0 mg/kg).

Animals were sacrificed at either 4 or 24 hours post LPS exposure via an intraperitoneal injection of euthansol. Blood was collected via cardiac stick (EDTA) immediately after euthanasia solution was administered and no reflexes were observed, and lung, BALF, and plasma samples were then harvested. Lung samples were weighed, lavaged twice with 5 mL of Dulbecco's phosphate buffered (PBS) solution, and homogenized prior to being assayed for erythromycylamine concentration and cytokine levels (inflammatory endpoints). The BALF was centrifuged, supernatants were removed, and the cellular pellet was resuspended in 1 mL of PBS. Total cell counts and differentials were determined by manual count according to LRRI SOPs.

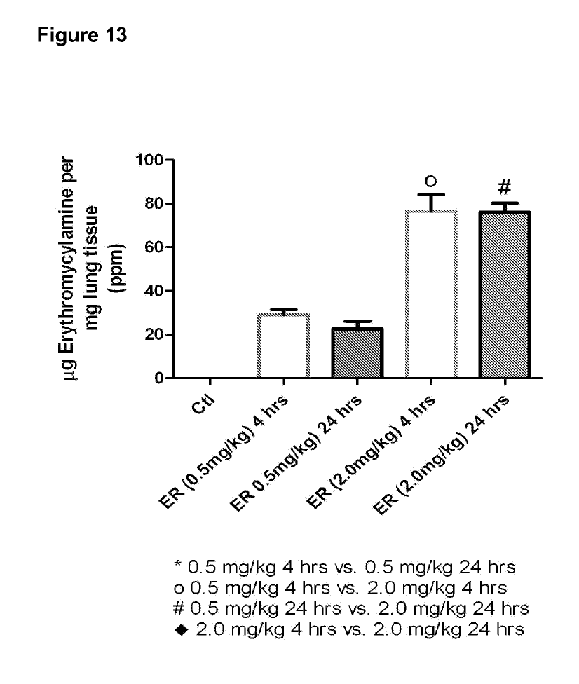

FIG. 13 shows the average and standard deviation of lung tissue concentrations from the control, 0.5 and 2.0 mg/kg treatment groups. Erythromycylamine lung concentrations were approximately 20 and 80 ppm at the 0.5 and 2.0 mg/kg doses. The concentrations showed good dose proportionality between the two dose groups, and there was no decrease in tissue concentration between 4 and 24 hr. Assay performance was verified for linearity accuracy and precision. Table 7 shows a summary of the theoretical versus measured concentration of erythromycylamine spiked into lung tissues at concentrations spanning 474 ng/ml to 30.369 ng/ml. As shown, the measured values were all within 15% of theoretical.

TABLE 7

| Theoretical | Average Measured | Percent |
|---|---|---|
| Conc. (ng/ml) | Conc. (ng/ml) | Accuracy |
| 474.54 | 477.05 | 101 |
| 1898.13 | 1,736.41 | 91.5 |
| 7592.49 | 7,559.86 | 100 |
| 24,295.94 | 27,700.77 | 114 |
| 30,369.91 | 29,679.86 | 97.7 |

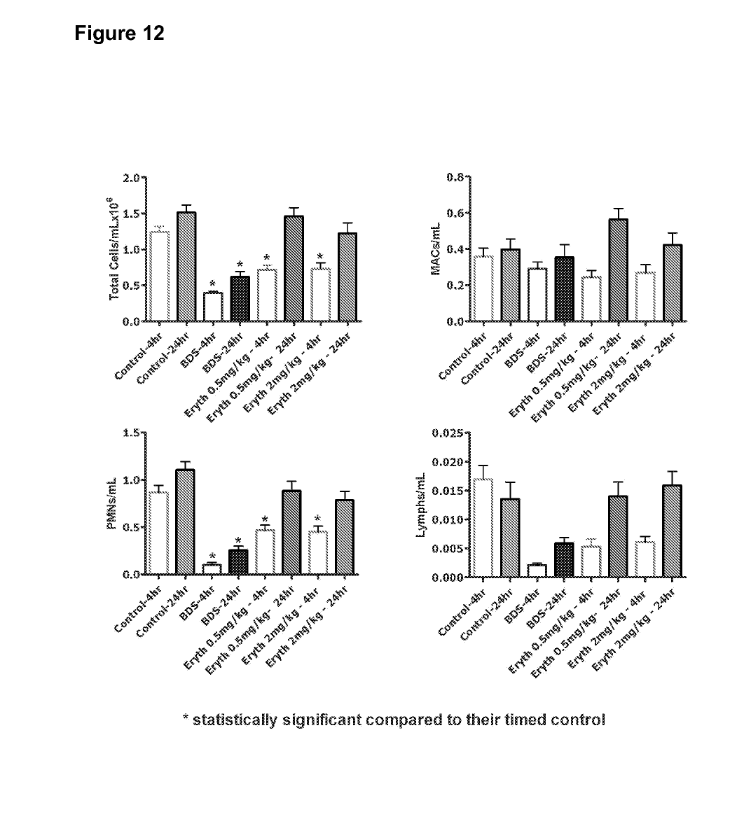

FIG. 12 shows cell counts and differentials for BALF samples. Statistically significant differences from control (matched to each respective time point) are indicated at p=0.05. Both the high and low doses of erythromycylamine significantly inhibited the total cell count and neutrophils 4 hours post LPS exposure, but did not show significant inhibition at 24 hours post exposure. Budesonide reduced inflammation 4 and 24 hours post LPS exposure.

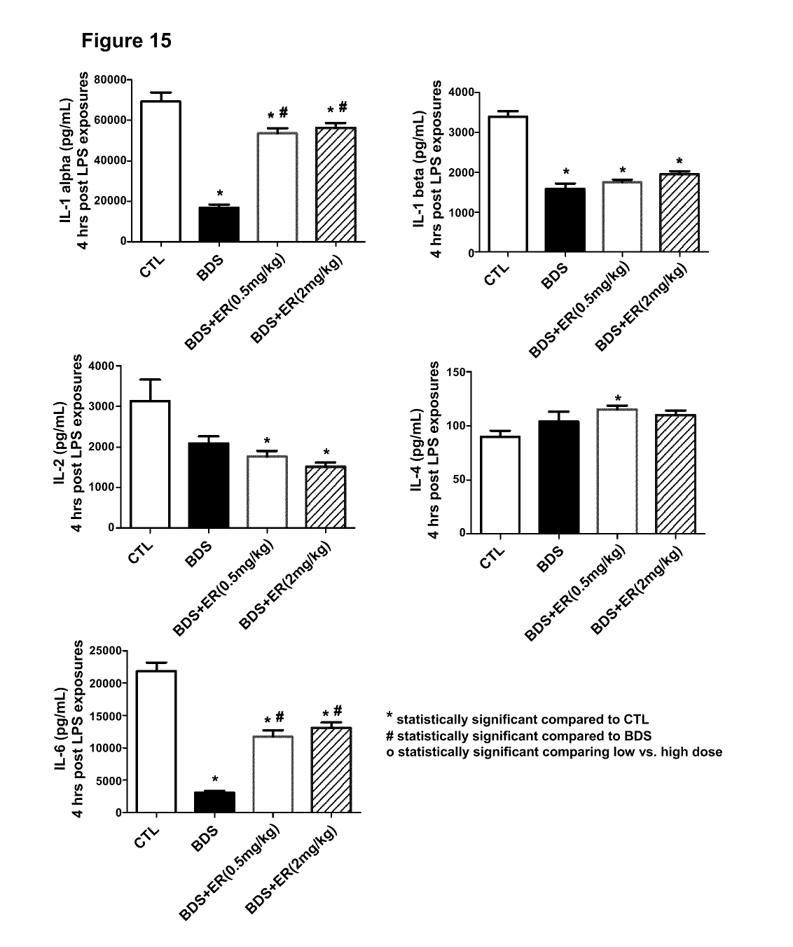

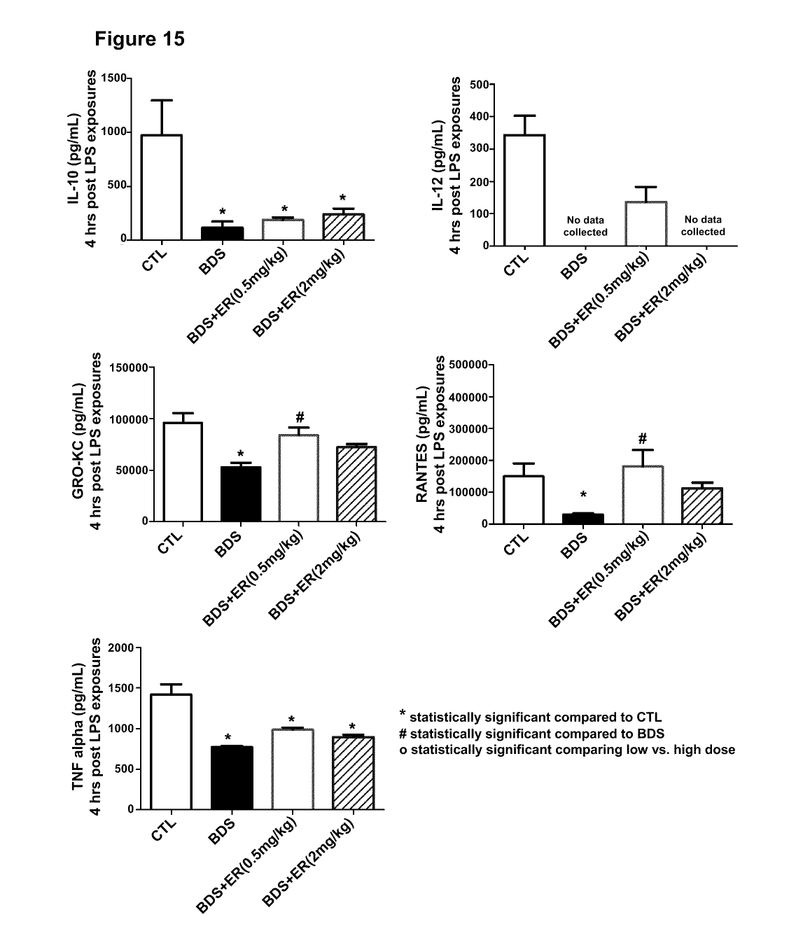

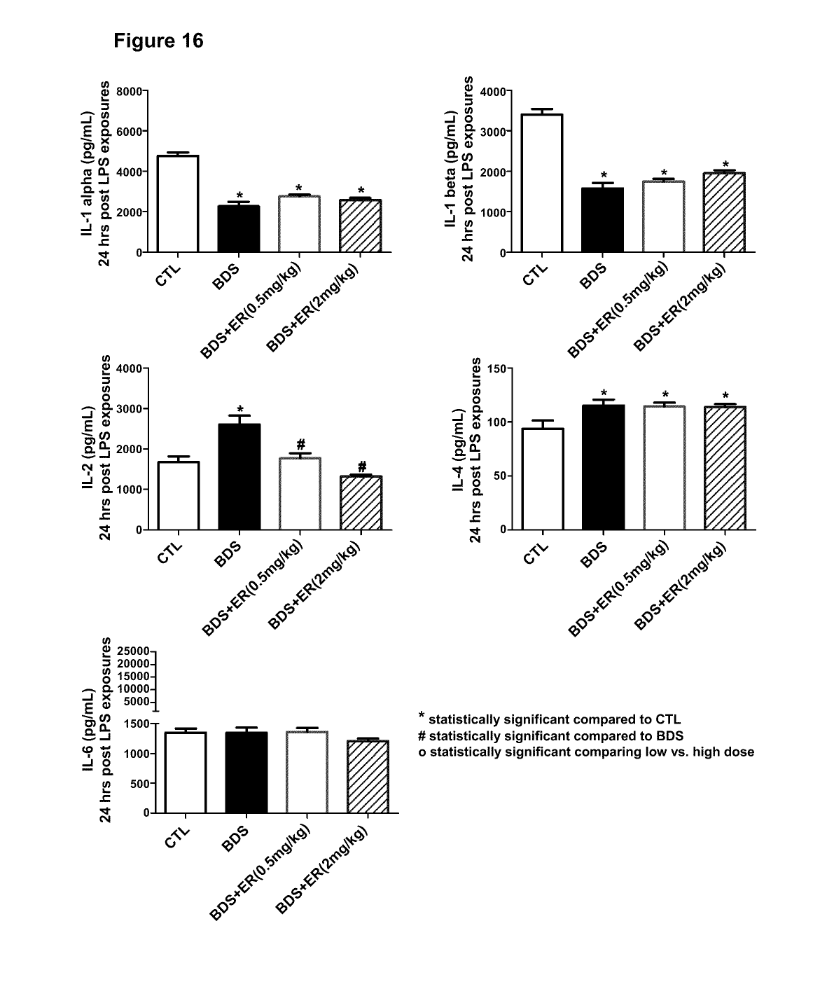

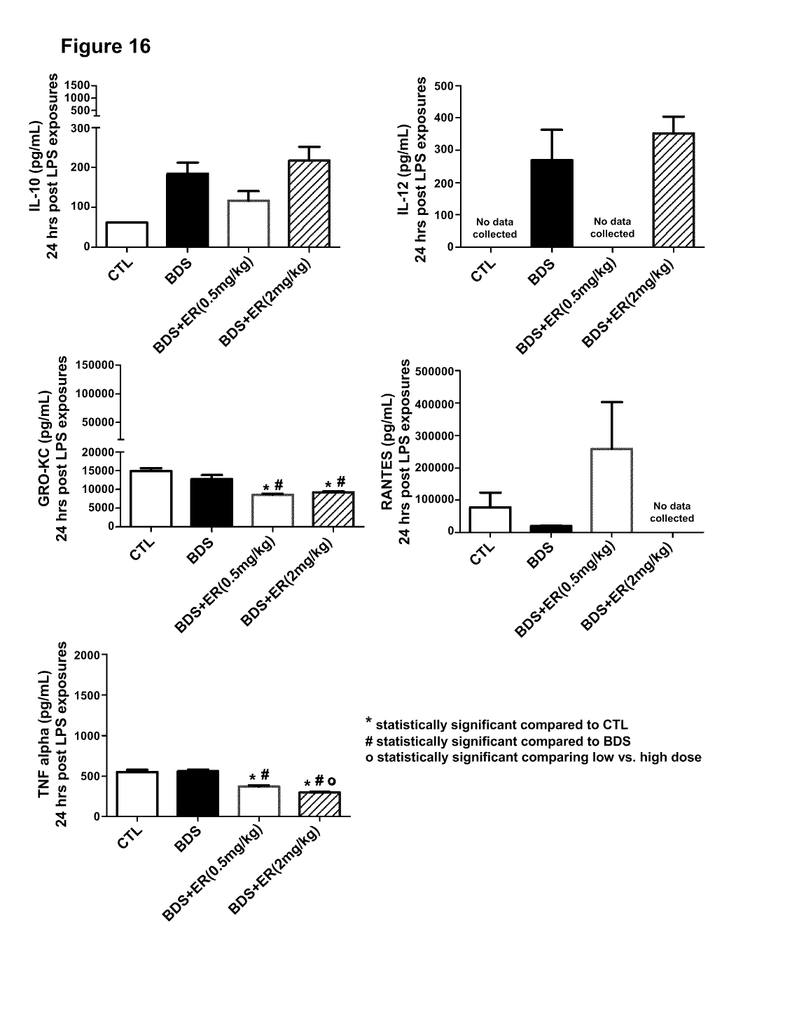

Results of the cytokine analysis are shown in FIGS. 15-16. Samples were analyzed by the bead-based immunoassay system using Lincoplex reagents (Millipore) for the following analytes: IL-1α, IL-1β, IL-2, IL-4, IL-6, IL-10, IL-12, GRO-KC, RANTES, TNF-alpha, IFN-gamma, and GM-CSF. Following incubation with the beads, washing, addition of the fluorescent secondary reagent, and additional washing, the cytokines were quantified using a BioPlex 100 (BioRad, Carlsbad, Calif.) analysis system, according to the recommendation of the supplier of the instrument. Results were statistically analyzed for differences versus control or animals treated with Budesonide Sulfate.

Statistically significant changes from control were observed in the presence of erythromycylamine at both 4 and 24 hours post LPS exposure for IL-1α, IL-1β, IL-2, IL-4, and TNF-α. Statistically significant changes from control were observed in the presence of erythromycylamine at 4 hours post LPS exposure for IL-6 and IL-10, and at 24 hours post LPS exposure for GRO-KC. No differences were observed for IL-12. For both IL-12 and RANTES no data were collected for some of the groups because the results were below the range for the assay. All samples for IFN-gamma and GMCSF were below the range of the assay, and are thus not reported.

### CONCLUSIONS

Aerosolized erythromycylamine is effective in inhibiting lung inflammation in the F344 Rat LPS inflammation model. Inhibition of neutrophils influx and cytokine expression was observed at lung concentrations of 20 and 80 ppm (subsequent to doses of 0.5 and 2.0 mg/kg administered 12 and 48 hrs prior to LPS). Inflammation (neutrophils) reduction was similar at both doses, but the effect subsided by 24 hr. In most cases the cytokine expression was effected at both time points. The degree of inflammation reduction was not dose dependent, suggesting the pharmacodynamic effect is saturated at the lower dose.

The invention being thus described, it will be obvious that the same can be varied in many ways. Such variations are not to be regarded as a departure from the spirit and scope of the invention, and all such modifications as would be obvious to one skilled in the art are intended to be included within the scope of the following claims.

What is claimed:

1. A method for treating inflammation in the lungs of a subject comprising administering an effective amount of an aerosolized macrolide antibiotic to the subject by inhalation, the antibiotic being administered with a dosing interval of at least 5 days.

2. The method of claim 1, wherein the effective amount treats the inflammation throughout the dosing interval without systemic side effects.

3. The method of claim 1, wherein the effective amount treats the inflammation throughout the dosing interval without nausea, vomiting, diarrhea, abdominal pain, vomiting, or dyspepsia.

4. The method of claim 1, wherein the method is carried out for at least 3 months.

5. The method of claim 1, wherein the dosing interval is at least 7 days.

6. The method of claim 1, further comprising administering a loading dose of an aerosolized macrolide antibiotic to the subject by inhalation, the loading dose being administered with a dosing interval of less than 5 days.

7. The method of claim 6, wherein the dosing interval between loading doses is between about 12 and 72 hours.

8. The method of claim 6, wherein the loading dose is greater than the effective dose.

9. The method of claim 1, wherein the inflammation is induced by or associated with elevated levels of one or more cytokines in the lungs.

10. The method of claim 9, wherein the inflammation is induced by or associated with elevated levels of a cytokine selected from the group consisting of: interleukin-1α (IL-1α), interleukin-1β (IL-1β), interleukin-8 (IL-8), interleukin-17 (IL-17), TNF-α, interferon-gamma (IFN-γ) and tissue growth factor 3 (TGF-β3).

11. The method of claim 1, wherein the inflammation is induced by or associated with increased levels of one or more inflammatory cells in the lungs.

12. The method of claim 11, wherein the one or more inflammatory cells are selected from the group consisting of: eosinophils, lymphocytes, macrophages, neutrophils and monocytes.

13. The method of claim 1, wherein the inflammation is associated with a condition selected from the group consisting of asthma, allergic asthma, emphysema, inflammatory lung injury, bronchiolitis obliterans (BO), pulmonary sarcoisosis, chronic obstructive pulmonary disease (COPD), interstitial lung disease, idiopathic pulmonary fibrosis, adult respiratory distress syndrome (ARDS), bronchiectasis, lung eosinophilia, interstitial fibrosis, and cystic fibrosis (CF).

14. The method of claim 1, wherein the inflammation is associated with transplantation of an organ, tissue and/or cells to the subject.

15. The method of claim 1, which is effective in elevating levels of an anti-inflammatory cytokine in the lungs of the subject throughout the dosing interval.

16. The method of claim 15, wherein the anti-inflammatory cytokine is selected from interleukin-4 (IL-4) and interleukin-10.

17. The method of claim 1, which is effective in reducing levels of a pro-inflammatory cytokine in the lungs of the subject throughout the dosing interval.

18. The method of claim 17, wherein the pro-inflammatory cytokine is selected from the group consisting of: interleukin-1α (IL-1α), interleukin-1β (IL-1β), interleukin-8 (IL-8), interleukin 17 (IL-17), TNF-α, interferon-gamma (IFN-γ) and tissue growth factor β (TGF-β).

19. The method of claim 1, wherein the macrolide antibiotic is erythromycylamine.

20. The method of claim 19, wherein the average minimum concentration (Cmin) of erythromycylamine during the dosing interval is at least 1.0 mg/kg.

21. The method of claim 19, wherein the average minimum concentration (Cmin) of erythromycylamine during the dosing interval is at least 0.5 mg/kg in the lung tissue of the subject.

22. The method of claim 19, wherein the average minimum concentration (Cmin) of erythromycylamine during the dosing interval is at least 0.1 mg/kg in the lung tissue of the subject.

23. The method of claim 19, wherein the average Cmin of erythromycylamine during the dosing interval is at least 50 times greater in the lung tissue of the subject than in the plasma, serum, and/or peripheral tissues of the subject.

24. The method of claim 19, wherein the average Cmin of erythromycylamine during the dosing interval is at least 100 times greater in the lung tissue of the subject than in the plasma, serum, and/or peripheral tissues of the subject.

25. The method of claim 19, wherein the average maximum concentration (Cmax) of erythromycylamine during the dosing interval is less than 0.1 mg/kg in the plasma of the subject.

26. The method of claim 19, wherein the average maximum concentration (Cmax) of erythromycylamine during the dosing interval is less than 0.5 mg/kg in the plasma of the subject.

27. The method of claim 19, wherein the terminal half-life of erythromycylamine in the lungs of the subject is at least 48 hours.

28. The method of claim 27, wherein the terminal half-life of erythromycylamine in the lungs of the subject is at least 96 hours.

29. A method for reducing or inhibiting inflammation in the lungs of a subject comprising administering an anti-inflammatory composition to the subject by inhalation, the composition comprising a combination of a macrolide antibiotic and cyclosporine in a pharmaceutically acceptable carrier suitable for aerosolized drug delivery.

30. The method of claim 29, wherein the composition has a greater than additive effect on reducing or inhibiting inflammation in the lungs.

31. A pharmaceutical composition comprising a combination of a macrolide antibiotic and cyclosporine in a pharmaceutically acceptable carrier suitable for aerosolized drug delivery, wherein the combination has a greater than additive effect on reducing or inhibiting inflammation in the lungs when administered by inhalation.

32. A method for treating inflammation in the lungs of a subject, comprising administering to the subject by inhalation a low dose of an aerosolized macrolide antibiotic, the low dose being less than 50 mg and effective to treat the inflammation without systemic side effects.

33. The method of claim 32, wherein the macrolide antibiotic is erythromycylamine.

34. The method of claim 32, wherein the low dose is effective to treat the inflammation without nausea, vomiting, diarrhea, abdominal pain, vomiting, or dyspepsia.

35. The method of claim 32, wherein the inflammation is induced by or associated with elevated levels of one or more cytokines in the lungs.

36. The method of claim 32, wherein the inflammation is induced by or associated with elevated levels of a cytokine selected from the group consisting of: interleukin-1α (IL-1α), interleukin-1β (IL-1β), interleukin-8 (IL-8), interleukin-17 (IL-17), TNF-α, interferon-gamma (IFN-γ) and tissue growth factor β (TGF-β).

37. The method of claim 32, wherein the inflammation is associated with a condition selected from the group consisting of asthma, allergic asthma, emphysema, inflammatory lung injury, bronchiolitis obliterans (BO), pulmonary sarcoisosis, chronic obstructive pulmonary disease (COPD), interstitial lung disease, idiopathic pulmonary fibrosis, adult respiratory distress syndrome (ARDS), bronchiectasis, lung eosinophilia, interstitial fibrosis, and cystic fibrosis (CF).

38. The method of claim 32, wherein the inflammation is associated with transplantation of an organ, tissue and/or cells to the subject.

39. The method of claim 32, wherein the inflammation is induced by or associated with increased levels of one or more inflammatory cells in the lungs.

40. The method of claim 39, wherein the one or more inflammatory cells are selected from the group consisting of: eosinophils, lymphocytes, macrophages, neutrophils and monocytes.

41. The method of claim 32, which is effective in elevating levels of an anti-inflammatory cytokine in the lungs of the subject.

42. The method of claim 41, wherein the anti-inflammatory cytokine is selected from interleukin-4 (IL-4) and interleukin-10.

43. The method of claim 32, which is effective in reducing levels of a pro-inflammatory cytokine in the lungs of the subject.

44. The method of claim 43, wherein the pro-inflammatory cytokine is selected from the group consisting of: interleukin-1α (IL-1α), interleukin-1β (IL-1β), interleukin-8 (IL-8), TNF-α, interferon-gamma (IFN-γ) and tissue growth factor β (TGF-β).
